(19) United States
(12) Patent Application Publication
(10) Pub. No.: US 2024/0035077 A1
(43) Pub. Date: Feb. 1, 2024

(54) METHODS FOR PERFORMING MULTIPLEXED REAL-TIME PCR WITH THE USE OF LARGE STOKES SHIFT FLUORESCENT DYES

(71) Applicant: Roche Molecular Systems, Inc., Pleasanton, CA (US)

(72) Inventors: Alexander Nierth, Dublin, CA (US); Fangnian Wang, Millis, MA (US); Jin Wang, Bedford, MA (US)

(21) Appl. No.: 18/258,872

(22) PCT Filed: Dec. 21, 2021
(86) PCT No.: PCT/EP2021/087165

§ 371 Date: Jun. 22, 2023

Related U.S. Application Data
(60) Provisional application No. 63/129,423, filed on Dec. 22, 2020.

(51) Int. Cl. C12Q 1/686

(52) U.S. Cl. CPC C12Q 1/686

(57) ABSTRACT

The present invention allows for the expansion of multiplexing capabilities of common PCR devices by using fluorogenic PCR probes made of large Stokes shift (LSS) fluorescent dyes. With this approach, no changes of the hardware or software components in the instrument are required.

### FIELD OF THE INVENTION

The present invention relates to methods for polymerase chain reaction (PCR), particularly to methods for performing multiplexed real-time PCR using large stokes shift fluorescent dyes.

### BACKGROUND OF THE INVENTION

The polymerase chain reaction (PCR) has become a ubiquitous tool of biomedical research, disease monitoring and diagnostics. Amplification of nucleic acid sequences by PCR is described in U.S. Pat. Nos. 4,683,195, 4,683,202, and 4,965,188. PCR is now well known in the art and has been described extensively in the scientific literature. See PCR Applications, ((1999) Innis et al., eds., Academic Press, San Diego), PCR Strategies, ((1995) Innis et al., eds., Academic Press, San Diego); PCR Protocols, ((1990) Innis et al., eds., Academic Press, San Diego), and PCR Technology, ((1989) Erlich, ed., Stockton Press, New York). A “real-time” PCR assay is able to simultaneously amplify and detect and/or quantify the starting amount of the target sequence. The basic TaqMan real-time PCR assay using the 5′-to-3′ nuclease activity of the DNA polymerase is described in Holland et al., (1991) Proc. Natl. Acad. Sci. 88:7276-7280 and U.S. Pat. No. 5,210,015. A real-time PCR without the nuclease activity (a nuclease-free assay) has been described in U.S. Patent Publication No. 20100143901A1. The use of fluorescent probes in real-time PCR is described in U.S. Pat. No. 5,538,848.

A typical real-time PCR protocol with fluorescent probes involves the use of a labeled probe, specific for each target sequence. The probe is preferably labeled with one or more fluorescent moieties, which absorb and emit light at specific wavelengths. Upon hybridizing to the target sequence or its amplicon, the probe exhibits a detectable change in fluorescent emission as a result of probe hybridization or hydrolysis.

The major challenge of the real-time assay however remains the ability to analyze numerous targets in a single tube. In virtually every field of medicine and diagnostics, the number of loci of interest increases rapidly. For example, multiple loci must be analyzed in forensic DNA profiling, pathogenic microorganism detection, multi-locus genetic disease screening and multi-gene expression studies, to name a few.

With the majority of current methods, the ability to multiplex an assay is limited by the detection instrument. Specifically, the use of multiple probes in the same reaction requires the use of distinct fluorescent labels. To simultaneously detect multiple probes, an instrument must be able to discriminate among the light signals emitted by each probe. The majority of current technologies on the market do not permit detection of more than four to seven separate wavelengths in the same reaction vessel. Therefore, using one uniquely labeled probe per target, no more than four to seven separate targets can be detected in the same vessel. In practice, at least one target is usually a control nucleic acid. Accordingly, in practice, no more than three to six experimental targets can be detected in the same tube. The use of fluorescent dyes is also limited due to the spectral bandwidth where only about six or seven dyes can be fit within the visible spectrum without significant overlap interference. Thus, the ability to multiplex an assay will not keep pace with the clinical needs, unless radical changes in the amplification and detection strategies are made. An additional ability to multiplex a real-time amplification reaction is provided by a post-PCR melting assay. See U.S. Patent Publication No. 20070072211A1. In a melting assay, the amplified nucleic acid is identified by its unique melting profile. A melting assay involves determining the melting temperature (melting point) of a double-stranded target, or a duplex between the labeled probe and the target. As described in U.S. Pat. No. 5,871,908, to determine melting temperature using a fluorescently labeled probe, a duplex between the target nucleic acid and the probe is gradually heated (or cooled) in a controlled temperature program. The dissociation of the duplex changes the distance between interacting fluorophores or between fluorophore and quencher. The interacting fluorophores may be conjugated to separate probe molecules, as described in U.S. Pat. No. 6,174,670. Alternatively, one fluorophore may be conjugated to a probe, while the other fluorophore may be intercalated into a nucleic acid duplex, as described in U.S. Pat. No. 5,871,908. As yet another alternative, the fluorophores may be conjugated to a single probe oligonucleotide. Upon the melting of the duplex, the fluorescence is quenched as the fluorophore and the quencher are brought together in the now single-stranded probe.

The melting of the nucleic acid duplex is monitored by measuring the associated change in fluorescence. The change in fluorescence may be represented on a graph referred to as “melting profile.” Because different probe-target duplexes may be designed to melt (or reanneal) at different temperatures, each probe will generate a unique melting profile. Properly designed probes would have melting temperatures that are clearly distinguishable from those of the other probes in the same assay. Many existing software tools enable one to design probes for a same-tube multiplex assay with these goals in mind. For example, Visual OMP™ software (DNA Software, Inc., Ann Arbor, Mich.) enables one to determine melting temperatures of nucleic acid duplexes under various reaction conditions.

The method of multiplex PCR using fluorescence detection and a subsequent post-amplification melting assay is described in U.S. Pat. No. 6,472,156. The number of targets detectable by such a method is a product of the number of detectable wavelengths and the number of distinguishable melting profiles. Therefore adding a melting assay to color detection was a step forward in the ability to detect multiple targets.

The post-amplification melting assay is most commonly used for qualitative purposes, i.e. to identify target nucleic acids, see U.S. Pat. Nos. 6,174,670; 6,427,156; and 5,871,908. It is known to obtain a melting peak by differentiating the melting curve function. Ririe et al. (“Product differentiation by analysis of DNA melting curves during the polymerase chain reaction,” (1997) Anal. Biochem. 245:154-160) observed that differentiation helps resolve melting curves generated by mixtures of products. After differentiation, the melting peaks generated by each component of the mixture become easily distinguishable. It was also previously known that the post-amplification melting signal, i.e. melting peak, is higher in proportion to the amount of the nucleic acid in the sample. For example, U.S. Pat. No. 6,245,514 teaches a post-amplification melt assay using a duplex-intercalating dye, to generate a derivative melting peak, and then, using proprietary software, to integrate the peak. The integration provides information about the efficiency of amplification and relative amount of the amplified nucleic acid.

In practice, it would be desirable to move beyond a qualitative assay and be able to quantify multiple targets in the same sample. See e.g. Sparano et al. “Development of the 21-gene assay and its application in clinical practice and clinical trials,” J. Clin. Oncol. (2008) 26(5):721-728. The ability to quantify the amount of target is useful in clinical applications, such as determination of viral load in a patient's serum, measuring the level of expression of a gene in response to drug therapy, or determining the molecular signature of a tumor to predict its response to therapy.

In a real-time PCR assay, the signal generated by the labeled probe can be used to estimate the amount of input target nucleic acid. The greater the input, the earlier the fluorescence signal crosses a predetermined threshold value (Ct). Therefore, one can determine relative or absolute amounts of the target nucleic acid by comparing the samples to each other or to a control sample with known amount of nucleic acid. However, the existing methods are limited in their ability to simultaneously quantify multiple targets. As with the qualitative detection of multiple targets, the limiting factor is the availability of spectrally resolvable fluorophores. As explained above, state-of-the-art fluorescent label technology is not able to obtain distinct signals from more than six or seven separate fluorescently labeled probes in the same tube. Therefore, a radically different experimental approach is needed to permit amplification and detection of numerous nucleic acid targets during real-time PCR.

Commercial fluorescence-based devices for automated polymerase chain reaction (PCR) can detect multiple targets in a single reaction vessel (multiplexing) by distinguishing light from differently colored fluorophores. The selection of dyes is characterized by minimizing their spectral overlap. Every fluorophore in the ensemble is excited with light at or near the absorption maximum and the emitted light (fluorescence) is detected at or near the fluorescence maximum. By limiting the range of wavelengths (band) for excitation and emission with optical filters, individual fluorophores can be distinguished. The specific combination of an excitation band and a simultaneously detected emission band defines an optical channel, each allowing for the identification of one PCR target. The achievable maximum number of optical channels depends on numerous interrelated factors, such as available spectral range, excitation light intensity, fluorophore brightness, fluorophore spectral width, filter bandwidth, and detector sensitivity. State-of-the-art PCR devices with fluorescence-based detection technology use between four and up to six optical filters per excitation and emission pathway. Therefore, with standard fluorophores, four to six PCR targets can be distinguished. The present invention allows expanding the multiplexing capabilities of common PCR devices by using fluorogenic PCR probes made of large Stokes shift (LSS) fluorescent dyes. With this approach, no changes of the hardware or software components in the instrument are required.

### SUMMARY OF THE INVENTION

The present invention relates to the use of fluorescent dyes with large Stokes shift (LSS) for increasing the number of simultaneously detectable targets in a single reaction vessel (multiplexing) during polymerase chain reaction (PCR) and is defined in the appended claims. Although LSS dyes have been used in molecular imaging, cellular imaging, tissue imaging, and as control reference dyes in PCR reactions, they have not been used for the specific purpose of expanding the multiplexing capacity of PCR instruments designed for fluorescent signal detection in real-time PCR.

Therefore, in one aspect, the present invention provides for a method for detecting at least two target nucleic acid sequences in a sample comprising the steps of: (a) contacting said sample suspected of containing said at least two target nucleic acid sequences in a single reaction vessel with: i. a first pair of oligonucleotide primers with nucleotide sequences that are complementary to each strand of a first target nucleic acid sequence, and a second pair of oligonucleotide primers with nucleotide sequences that are complementary to each strand of a second target nucleic acid sequence; ii. a first oligonucleotide probe comprising a nucleotide sequence at least partially complementary to the first target nucleic acid sequence and that anneals within the first target nucleic acid sequence bounded by the first pair of oligonucleotide primers, wherein said first oligonucleotide probe is labeled with a large stokes shift (LSS) fluorescent dye capable of generating a detectable signal, and with a first quencher moiety capable of quenching the detectable signal generated by the LSS fluorescent dye, wherein the LSS fluorescent dye is separated from the first quencher moiety by a nuclease susceptible cleavage site; iii a second oligonucleotide probe comprising a nucleotide sequence at least partially complementary to the second target nucleic acid sequence and that anneals within the second target nucleic acid sequence bounded by the second pair of oligonucleotide primers, wherein said second oligonucleotide probe is labeled with a small stokes shift (SSS) fluorescent dye capable of generating a detectable signal, and with a second quencher moiety capable of quenching the detectable signal generated by the SSS fluorescent dye, wherein the SSS fluorescent dye is separated from the second quencher moiety by a nuclease susceptible cleavage site, and wherein the SSS fluorescent dye has an absorption peak maximum significantly different from and an emission peak maximum similar to respective peak maxima of the LSS fluorescent dye on the first oligonucleotide probe, wherein the significant difference is at least 80 nanometer in wavelength; (b) amplifying the first and second target nucleic acid sequences by polymerase chain reaction (PCR) using a nucleic acid polymerase having 5′ to 3′ nuclease activity such that during an extension step of each PCR cycle, the 5′ to 3′ nuclease activity of the nucleic acid polymerase allows cleavage and separation of the LSS fluorescent dye from the first quenching moiety on the first oligonucleotide probe, and cleavage and separation of the SSS fluorescent dye from the second quenching moiety on the second oligonucleotide probe; (c) measuring the detectable signal from the LSS fluorescent dye by excitation at or near the wavelength of the absorption peak maximum of the LSS fluorescent dye and measuring the detectable signal from the SSS fluorescent dye by excitation at or near the wavelength of the absorption peak of the SSS fluorescent dye; (d) repeating steps (b) and (c) in multiple PCR cycles to produce desired quantity of amplification products from the first and second target nucleic acid sequences; (e) detecting the presence of the first target nucleic acid sequence from the signals detected from the LSS fluorescent dye and the presence of the second target nucleic acid sequence from the signals detected from the SSS fluorescent dye.

In one embodiment, the SSS fluorescent dye on the second oligonucleotide probe has an emission peak maximum significantly different from and an absorption peak maximum similar to respective peak maxima of the LSS fluorescent dye on the first oligonucleotide probe, wherein the significant difference is at least 80 nanometer in wavelength. In another embodiment, the difference between the absorption peak maximum of the LSS fluorescent dye and the absorption peak maximum of the SSS fluorescent dye is greater than 80 nanometers in wavelength. In yet another embodiment, the difference is greater than 100 nanometer in wavelength. In a further embodiment, the difference between the emission peak maximum of the LSS fluorescent dye and the emission peak maximum of the SSS fluorescent dye is greater than 80 nanometers in wavelength. In yet another embodiment, the difference is greater than 100 nanometer in wavelength.

In one embodiment, the LSS fluorescent dye is selected from the group consisting of: ALEXA FLUOR 430, ATTO 430LS, ATTO 490LS, ATTO 390LS, CASCADE YELLOW, CF350, CHROMEO 494, CYTO 500 LSS, CYTO 510 LSS, CYTO 514 LSS, CYTO 520 LSS, DAPOXYL, DY 480XL, DY 481XL, DY 485XL, DY 510XL, DY 511XL, DY 520XL, DY 521XL, DY 601XL, DY 350XL, DY 360XL, DY 370XL, DY 375XL, DY 380XL, DY 395XL, DY 396XL, DYLIGHT 515-LS, DYLIGHT 485-LS, DYLIGHT 510-LS, DYLIGHT 521-LS, FURA 2, INDO 1, KROME ORANGE, LUB 04, LUCIFER YELLOW, NBD X, NILE RED, PULSAR 650, PYMPO, STAR 440SXP, STAR 470SXP, STAR 520SXP, VIOGREEN, CF 350, SETAU 405, and PACIFIC ORANGE. In a further embodiment, the LSS fluorescent dye is DY396XL. In yet a further embodiment, the LSS fluorescent dye is CHROMEO 494. In another embodiment, the LSS fluorescent dye is ATTO 490LS.

In one embodiment, the LSS fluorescent dye has a fluorescence signal strength that remains stable at temperatures up to 100° C. In one embodiment, the LSS fluorescent dye is ATTO 490LS. In another embodiment, the first oligonucleotide probe, the second oligonucleotide probe or both the first oligonucleotide probe and the second oligonucleotide probe is a tagged probe compatible with the TAGS technology.

In another embodiment, the methods of the present invention are conducted in a single reaction vessel that is a tubule comprising (i) a proximal end having an opening through which a sample is introducible; (ii) a distal end; and (iii) at least a first segment containing at least one nucleic acid extraction reagent, a second segment distal to the first segment and containing a wash reagent, and a third segment distal to the second segment and containing one or more amplification reagents, each of the segments being (A) defined by the tubule; (B) fluidly isolated, at least in part, by a fluid-tight seal formed by a bonding of opposed wall portions of the tubule to one another such that (1) the seal is broken by application of fluid pressure on a segment that is fluidly isolated in part by the seal; and (2) the seal is capable of being clamped where the opposed wall portions of the tubule are bonded, without breaking the seal, to prevent the seal from being broken by application of fluid pressure on a segment that is fluidly isolated in part by the seal; (C) so expandable as to receive a volume of fluid expelled from another segment; and so compressible as to contain substantially no fluid when so compressed; (iv) a cap for closing the opening, the cap containing a chamber in fluid communication with the tubule, and the cap permitting free escape of gasses but retaining all liquid volumes and infectious agents in the tube; (v) a rigid frame to which the tubule's proximal and distal ends are held; and (vi) an integral tubule tensioning mechanism or an attachment of the tubule to the frame that pulls the tubule sufficiently taut so as to facilitate compression and flattening of the tubule.

### BRIEF DESCRIPTION OF THE DRAWINGS

FIG. 1. Channel assignment matrix for a six-color PCR instrument. Conceptual representation on how the conventional optical channels of a six-color PCR instrument can be extended by new combinations of excitation and emission filters, leading to twenty-one possible detection channels. The center wavelength for the excitation and emission filters are indicated in nanometers. The descriptors for excitation and emission are UV (u), blue (b), green (g), amber (a), red (r), IR, (i). An optical channel is created by combining one excitation and one emission filter. For example, the two-letter channel descriptor for excitation in the UV and emission in the green is “ug”. Channels highlighted in light gray correspond to conventional channels that are accessible with standard Short Stoke Shift (SSS) fluorophores. White fields correspond to channels that are accessible with currently available LSS dyes. Channels highlighted in dark gray are accessible by resonance electron transfer (RET) probes.

FIG. 2. Absorption (left) and emission spectra (right) for a six-color PCR instrument (Cobas® Liat® analyzer). Showcased are the spectra of four standard SSS fluorescent dyes and one LSS dye (Chromeo 494). Spectra were normalized to arbitrary absorption and fluorescence units (AU/FU). The wavelength regions covered by optical filters are indicated as horizontal lines. Chromeo 494 (dashed line), which has excitation/emission maxima at 494 nm/628 nm, can be excited with blue light and detected in the amber and/or red emission filters (ba and/or br-channel). FIG. 3. Absorption (left) and emission spectra (right) of a six-color PCR instrument (Cobas® Liat® analyzer). Showcased are the spectra of four standard SSS fluorescent dyes and one LSS dye (Dy396XL). Spectra were normalized to arbitrary absorption and fluorescence units (AU/FU). The wavelength regions covered by optical filters are indicated as horizontal lines. Dy396XL (dashed line), which has excitation/emission maxima at 392 nm/572 nm, can be excited with UV light and detected in the green emission filter (ug-channel).

FIG. 4. Real-time PCR growth curves of six TaqMan probes in the Cobas® Liat® CT/NG/TV/MG test with target input at 3×LoD level. Multiplexing in the Cobas® Liat® analyzer was achieved by combining four SSS dyes with two LSS dyes (Chromeo494, and Dy395XL). The baselines of the growth curves were normalized to zero for comparison.

FIG. 5. Statistical analysis of MGPB probes labeled with Dy395XL or Dy396XL in the 5-channel Cobas® Liat® CT/NG/MG test. Due to baseline difference between MG-Dy395XL and MG-Dy396XL probes, the performance (Ct, amplitude, and Kexp) of the probe with higher baseline was normalized according to the baseline ratio.

FIG. 6. Statistical analysis of MGPC probes labeled with Dy395XL or Dy396XL in 5-channel Cobas® Liat® CT/NG/MG test. Due to baseline difference between MG-Dy395XL and MG-Dy396XL probes, the performance (Ct, amplitude, and Kexp) of the probe with higher baseline was normalized according to the baseline ratio.

FIG. 7. Exemplary embodiment of a sample tube including a tubule for the Cobas® Liat® analyzer.

FIG. 7A is a front elevation view. FIG. 7B is a cross sectional view of a sample tube positioned inside an analyzer.

FIG. 8. Another exemplary embodiment of a sample tube including a tubule for the Cobas® Liat® analyzer. FIG. 8A is a cross sectional view. FIG. 8B is a perspective view of a sample tube.

FIG. 9. Optical channel assignment matrix for a five-color PCR instrument. Conceptual representation on how the conventional optical channels of a five-color PCR instrument can be extended by new combinations of excitation and emission filters. The center wavelength for the excitation and emission filters are indicated in nanometers. Optical channels on the diagonal fields (light gray) are accessible with standard Short Stoke Shift (SSS) fluorophores. Dark gray fields correspond to optical channels that were accessed with LSS dyes. In one embodiment, targets were detected with an LSS dye by excitation at 495 nm and detection at 645 nm (ATTO 490LS dye). In another embodiment, targets were detected with an LSS dye by excitation at 435 nm and detection at 580 nm (RLS dye). White fields correspond to further channels that are conceptually accessible with LSS dyes or resonance electron transfer (RET) probes.

FIG. 10. Absorption (left) and emission spectra (right) for five-color PCR instruments (LightCycler® and Cobas® x800 analyzers). Showcased are the spectra for five standard SSS fluorescent dyes and one LSS dye ATTO 490LS that can be detected by excitation at 495 nm and detection at 645 nm. Spectra were normalized to arbitrary absorption and fluorescence units (AU/FU). The wavelength regions covered by optical filters are indicated as horizontal lines.

FIG. 11. Absorption (left) and emission spectra (right) for five-color PCR instruments (LightCycler® and Cobas® x800 analyzers). Showcased are the spectra for five standard fluorescent dyes and one LSS dye (RLS) that can be detected by excitation at 435 nm and detection at 580 nm. Spectra were normalized to arbitrary absorption and fluorescence units (AU/FU). The wavelength regions covered by optical filters are indicated as horizontal lines.

FIG. 12. Real-time PCR growth curves that demonstrate the compatibility of extended optical multiplexing based on LSS dyes with temperature-based multiplexing. The featured example shows the individual detection of three targets in the ATTO 490LS optical channel (495 nm/645 nm) across three thermal channels (TC). TC1 is based on a standard TaqMan probe with ATTO 490LS and fluorescence readings at 58° C. TC2 and TC2 are based on tagged probe designs with ATTO 490LS that generate target fluorescence signals at 80° C. and 91° C. respectively. The target concentrations was 1000 cp/reaction in all samples were target was present. PCR growth curves for each thermal channel where a positive PCR signal was expected were marked with an asterisk. For the higher thermal channels, slight upward or downward slopes in the absence of target are caused by a non-optimized thermal correction factor of the fluorescent dye. The data was generated on a LightCycler® 480 analyzer.

FIG. 13. Real-time PCR growth curves that demonstrate the compatibility of extended optical multiplexing based on LSS dyes with temperature-based multiplexing. The featured example shows the individual detection of three targets in the RLS optical channel (435 nm/580 nm) across three thermal channels (TC). TC1 is based on a standard TaqMan probe with RLS dye and fluorescence readings at 58° C. TC2 and TC2 are based on tagged probe designs with the RLS dye that generate target fluorescence signals at 80° C. and 91° C. respectively. The concentrations of each target was 1000 cp/reaction, except for the no template control (NTC). For the higher thermal channels, slight upward or downward slopes in the absence of target are caused by a non-optimized thermal correction factor of the fluorescent dye. The data was generated on a LightCycler® 480 analyzer.

### DETAILED DESCRIPTION OF THE INVENTION

### Definitions

The term “sample” as used herein includes any specimen or culture (e.g., microbiological cultures) that includes nucleic acids. The term “sample” is also meant to include both biological and environmental samples. A sample may include a specimen of synthetic origin. Biological samples include whole blood, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), biopsy sample, urine, feces, sputum, saliva, nasal mucous, prostate fluid, semen, lymphatic fluid, bile, tears, sweat, breast milk, breast fluid, embryonic cells and fetal cells. In a preferred embodiment, the biological sample is blood, and more preferably plasma. As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The terms “target” or “target nucleic acid” as used herein are intended to mean any molecule whose presence is to be detected or measured or whose function, interactions or properties are to be studied. Therefore, a target includes essentially any molecule for which a detectable probe (e.g., oligonucleotide probe) or assay exists, or can be produced by one skilled in the art. For example, a target may be a biomolecule, such as a nucleic acid molecule, a polypeptide, a lipid, or a carbohydrate, which is capable of binding with or otherwise coming in contact with a detectable probe (e.g., an antibody), wherein the detectable probe also comprises nucleic acids capable of being detected by methods of the invention. As used herein, “detectable probe” refers to any molecule or agent capable of hybridizing or annealing to a target biomolecule of interest and allows for the specific detection of the target biomolecule as described herein. In one aspect of the invention, the target is a nucleic acid, and the detectable probe is an oligonucleotide. The terms “nucleic acid” and “nucleic acid molecule” may be used interchangeably throughout the disclosure.

The terms refer to oligonucleotides, oligos, polynucleotides, deoxyribonucleotide (DNA), genomic DNA, mitochondrial DNA (mtDNA), complementary DNA (cDNA), bacterial DNA, viral DNA, viral RNA, RNA, message RNA (mRNA), transfer RNA (tRNA), ribosomal RNA (rRNA), siRNA, catalytic RNA, clones, plasmids, M13, P1, cosmid, bacteria artificial chromosome (BAC), yeast artificial chromosome (YAC), amplified nucleic acid, amplicon, PCR product and other types of amplified nucleic acid, RNA/DNA hybrids and polyamide nucleic acids (PNAs), all of which can be in either single- or double-stranded form, and unless otherwise limited, would encompass known analogs of natural nucleotides that can function in a similar manner as naturally occurring nucleotides and combinations and/or mixtures thereof. Thus, the term “nucleotides” refers to both naturally occurring and modified/nonnaturally-occurring nucleotides, including nucleoside tri, di, and monophosphates as well as monophosphate monomers present within polynucleic acid or oligonucleotide. A nucleotide may also be a ribonucleotide; 2′-deoxynucleotide; or 2′,3′-deoxynucleotide as well as a vast array of other nucleotide mimics that are well-known in the art. Mimics include chain-terminating nucleotides, such as 3′-O-methyl, halogenated base or sugar substitutions; alternative sugar structures including nonsugar, alkyl ring structures; alternative bases including inosine; deaza-modified; chi- and/or psi-linker-modified; mass label-modified; phosphodiester modifications or replacements including phosphorothioate, methylphosphonate, boranophosphate, amide, ester, ether; and/or a basic or complete internucleotide replacements, including cleavage linkages such a photocleavable nitrophenyl moieties.

The presence or absence of a target can be measured quantitatively or qualitatively. Targets can come in a variety of different forms including, for example, simple or complex mixtures, or in substantially purified forms. For example, a target can be part of a sample that contains other components or can be the sole or major component of the sample. Therefore, a target can be a component of a whole cell or tissue, a cell or tissue extract, a fractionated lysate thereof or a substantially purified molecule. Also, a target can have either a known or unknown sequence or structure.

The term “amplification reaction” refers to any in vitro means for multiplying the copies of a target sequence of nucleic acid.

“Amplifying” refers to a step of submitting a solution to conditions sufficient to allow for amplification. Components of an amplification reaction may include, but are not limited to, e.g., primers, a polynucleotide template, polymerase, nucleotides, dNTPs and the like. The term “amplifying” typically refers to an “exponential” increase in target nucleic acid. However, “amplifying” as used herein can also refer to linear increases in the numbers of a select target sequence of nucleic acid, but is different from a one-time, single primer extension step.

“Polymerase chain reaction” or “PCR” refers to a method whereby a specific segment or subsequence of a target double-stranded DNA is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990.

“Oligonucleotide” as used herein refers to linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target nucleic acid. Usually monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG,” it will be understood that the nucleotides are in 5′-3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs, as noted above. Where an enzyme has specific oligonucleotide or polynucleotide substrate requirements for activity, e.g., single stranded DNA, RNA/DNA duplex, or the like, then selection of the appropriate composition for the oligonucleotide or polynucleotide substrates is well within the knowledge of one of ordinary skill.

As used herein “oligonucleotide primer”, or simply “primer”, refers to a polynucleotide sequence that hybridizes to a sequence on a target nucleic acid template and facilitates the detection of an oligonucleotide probe. In amplification embodiments of the invention, an oligonucleotide primer serves as a point of initiation of nucleic acid synthesis. In non-amplification embodiments, an oligonucleotide primer may be used to create a structure that is capable of being cleaved by a cleavage agent. Primers can be of a variety of lengths and are often less than 50 nucleotides in length, for example 12-25 nucleotides, in length. The length and sequences of primers for use in PCR can be designed based on principles known to those of skill in the art.

The term “oligonucleotide probe” as used herein refers to a polynucleotide sequence capable of hybridizing or annealing to a target nucleic acid of interest and allows for the specific detection of the target nucleic acid.

A “reporter moiety” or “reporter molecule” is a molecule that confers a detectable signal. The detectable phenotype can be colorimetric, fluorescent or luminescent, for example. Examples of fluorescent reporter moieties include, e.g., fluorescein (FAM), hexacholorofluorescein (HEX), JA270 (Roche Molecular Systems), cyanine dyes (e.g., CY3.5, CY5 or CY5.5).

A “quencher moiety” or “quencher molecule” is a molecule that is able to quench the detectable signal from the reporter moiety. Examples of quencher moieties used with fluorescent reporters include, e.g., the so-called dark quenchers, such as Black Hole Quenchers (BHQ-1 or BHQ-2) (LGC BioSearch Technologies) or Iowa Black (Integrated DNA Technologies); and fluorescent moities that use fluorescence resonance energy transfer (FRET), such as the cyanine dyes noted above.

A “mismatched nucleotide” or a “mismatch” refers to a nucleotide that is not complementary to the target sequence at that position or positions. An oligonucleotide probe may have at least one mismatch, but can also have 2, 3, 4, 5, 6 or 7 or more mismatched nucleotides.

The term “polymorphism” as used herein refers to an allelic variant. Polymorphisms can include single nucleotide polymorphisms (SNPs) as well as simple sequence length polymorphisms. A polymorphism can be due to one or more nucleotide substitutions at one allele in comparison to another allele or can be due to an insertion or deletion, duplication, inversion and other alterations known to the art.

The term “modification” as used herein refers to alterations of the oligonucleotide probe at the molecular level (e.g., base moiety, sugar moiety or phosphate backbone). Nucleoside modifications include, but are not limited to, the introduction of cleavage blockers or cleavage inducers, the introduction of minor groove binders, isotopic enrichment, isotopic depletion, the introduction of deuterium, and halogen modifications. Nucleoside modifications may also include moieties that increase the stringency of hybridization or increase the melting temperature of the oligonucleotide probe. For example, a nucleotide molecule may be modified with an extra bridge connecting the 2′ and 4′ carbons resulting in locked nucleic acid (LNA) nucleotide that is resistant to cleavage by a nuclease (as described in Imanishi et al., U.S. Pat. No. 6,268,490 and in Wengel et al., U.S. Pat. No. 6,794,499). The compositions of the tag portion of the oligonucleotide probe and of the quenching oligonucleotide molecule are only restricted by their ability to form stable duplexes. These oligonucleotides can therefore comprise of DNA, L-DNA, RNA, L-RNA, LNA, L-LNA, PNA (peptide nucleic acid, as described in Nielsen et al., U.S. Pat. No. 5,539,082), BNA (bridged nucleic acid, for example, 2′,4′-BNA(NC) [2′-O,4′-C-aminomethylene bridged nucleic acid] as described in Rahman et al., J. Am. Chem. Soc. 2008; 130(14):4886-96), L-BNA etc. (where the “L-XXX” refers to the L-enantiomer of the sugar unit of the nucleic acids) or any other known variations and modifications on the nucleotide bases, sugars, or phosphodiester backbones.

Other examples of nucleoside modifications include various 2′ substitutions such as halo, alkoxy and allyloxy groups that are introduced in the sugar moiety of oligonucleotides. Evidence has been presented that 2′-substituted-2′-deoxyadenosine polynucleotides resemble double-stranded RNA rather than DNA. Ikehara et al. (Nucleic Acids Res., 1978, 5, 3315) have shown that a 2′-fluro substituent in poly A, poly I, or poly C duplexed to its complement is significantly more stable than the ribonucleotide or deoxyribonucleotide poly duplex as determined by standard melting assays. Inoue et al. (Nucleic Acids Res., 1987, 15, 6131) have described the synthesis of mixed oligonucleotide sequences containing 2′-OMe (O-methyl) substituents on every nucleic nucleotide. The mixed 2′-OMe-substituted oligonucleotide hybridized to its RNA complement as strongly as the RNA-RNA duplex that is significantly stronger than the same sequence RNA-DNA heteroduplex. Examples of substitutions at the 2′ position of the sugar include F, CN, CF₃, OCF₃, OMe, OCN, O-alkyl, S-alkyl, SMe, SO₂Me, ONO₂, NO₂, NH₃, NH₂, NH-alkyl, OCH₃═CH₂ and OCCH.

The term “specific” or “specificity” in reference to the binding of one molecule to another molecule, such as a probe for a target polynucleotide, refers to the recognition, contact, and formation of a stable complex between the two molecules, together with substantially less recognition, contact, or complex formation of that molecule with other molecules. As used herein, the term “anneal” refers to the formation of a stable complex between two molecules. In particular, “anneal” can refer to formation of a stable double-stranded complex between complementary oligonucleotides.

A probe is “capable of annealing” to a nucleic acid sequence if at least one region of the probe shares substantial sequence identity with at least one region of the complement of the nucleic acid sequence. “Substantial sequence identity” is a sequence identity of at least about 80%, preferably at least about 85%, more preferably at least about 90%, 95% or 99%, and most preferably 100%. For the purpose of determining sequence identity of a DNA sequence and a RNA sequence, U and T often are considered the same nucleotide. For example, a probe comprising the sequence ATCAGC is capable of hybridizing to a target RNA sequence comprising the sequence GCUGAU. The term “cleavage agent” as used herein refers to any means that is capable of cleaving an oligonucleotide probe to yield fragments, including but not limited to enzymes. For methods wherein amplification does not occur, the cleavage agent may serve solely to cleave, degrade or otherwise separate the second portion of the oligonucleotide probe or fragments thereof. The cleavage agent may be an enzyme. The cleavage agent may be natural, synthetic, unmodified or modified.

For methods wherein amplification occurs, the cleavage agent is preferably an enzyme that possesses synthetic (or polymerization) activity and nuclease activity. Such an enzyme is often a nucleic acid amplification enzyme. An example of a nucleic acid amplification enzyme is a nucleic acid polymerase enzyme such as Thermus aquaticus (Taq) DNA polymerase or E. coli DNA polymerase I. The enzyme may be naturally occurring, unmodified or modified.

A “nucleic acid polymerase” refers to an enzyme that catalyzes the incorporation of nucleotides into a nucleic acid. Exemplary nucleic acid polymerases include DNA polymerases, RNA polymerases, terminal transferases, reverse transcriptases, telomerases and the like.

A “thermostable DNA polymerase” refers to a DNA polymerase that is stable (i.e., resists breakdown or denaturation) and retains sufficient catalytic activity when subjected to elevated temperatures for selected periods of time. For example, a thermostable DNA polymerase retains sufficient activity to effect subsequent primer extension reactions, when subjected to elevated temperatures for the time necessary to denature double-stranded nucleic acids. Heating conditions necessary for nucleic acid denaturation are well known in the art and are exemplified in U.S. Pat. Nos. 4,683,202 and 4,683,195. As used herein, a thermostable polymerase is typically suitable for use in a temperature cycling reaction such as the polymerase chain reaction (“PCR”). Examples of thermostable nucleic acid polymerases include Thermus aquaticus Taq DNA polymerase, Thermus sp. Z05 polymerase, Thermus flavus polymerase, Thermotoga maritima polymerases, such as TMA-25 and TMA-30 polymerases, Thermus thermophilus DNA polymerase, and the like.

A “modified” polymerase refers to a polymerase in which at least one monomer differs from the reference sequence, such as a native or wild-type form of the polymerase or another modified form of the polymerase. Such modified polymerases are described in, for example, US Patent Publication Nos. 20110294168A1 and 20140178911A1. Exemplary modifications include monomer insertions, deletions, and substitutions. Modified polymerases also include chimeric polymerases that have identifiable component sequences (e.g., structural or functional domains, etc.) derived from two or more parents. Also included within the definition of modified polymerases are those comprising chemical modifications of the reference sequence. The examples of modified polymerases include G46E E678G CS5 DNA polymerase, G46E L329A E678G CS5 DNA polymerase, G46E L329A D640G S671F CS5 DNA polymerase, G46E L329A D640G S671F E678G CS5 DNA polymerase, a G46E E678G CS6 DNA polymerase, Z05 DNA polymerase, ΔZ05 polymerase, ΔZ05-Gold polymerase, ΔZ05R polymerase, E615G Taq DNA polymerase, E678G TMA-25 polymerase, E678G TMA-30 polymerase, and the like.

The term “5′ to 3′ nuclease activity” or “5′-3′ nuclease activity” refers to an activity of a nucleic acid polymerase, typically associated with the nucleic acid strand synthesis, whereby nucleotides are removed from the 5′ end of nucleic acid strand, e.g., E. coli DNA polymerase I has this activity, whereas the Klenow fragment does not. Some enzymes that have 5′ to 3′ nuclease activity are 5′ to 3′ exonucleases. Examples of such 5′ to 3′ exonucleases include exonuclease from B. subtilis, phosphodiesterase from spleen, Lambda exonuclease, exonuclease II from yeast, exonuclease V from yeast, and exonuclease from Neurospora crassa.

Various aspects of the present invention are based on a special property of nucleic acid polymerases. Nucleic acid polymerases can possess several activities, among them, a 5′ to 3′ nuclease activity whereby the nucleic acid polymerase can cleave mononucleotides or small oligonucleotides from an oligonucleotide annealed to its larger, complementary polynucleotide. In order for cleavage to occur efficiently, an upstream oligonucleotide must also be annealed to the same larger polynucleotide.

The detection of a target nucleic acid utilizing the 5′ to 3′ nuclease activity can be performed by a “TaqMan® assay” or “5′-nuclease assay”, as described in U.S. Pat. Nos. 5,210,015; 5,487,972; and 5,804,375; and Holland et al., 1988, Proc. Natl. Acad. Sci. USA 88:7276-7280. In the TaqMan® assay, labeled detection probes that hybridize within the amplified region are present during the amplification reaction. The probes are modified so as to prevent the probes from acting as primers for DNA synthesis. The amplification is performed using a DNA polymerase having 5′ to 3′ exonuclease activity. During each synthesis step of the amplification, any probe that hybridizes to the target nucleic acid downstream from the primer being extended is degraded by the 5′ to 3′ exonuclease activity of the DNA polymerase. Thus, the synthesis of a new target strand also results in the degradation of a probe, and the accumulation of degradation product provides a measure of the synthesis of target sequences.

Any method suitable for detecting degradation product can be used in a 5′ nuclease assay. Often, the detection probe is labeled with two fluorescent dyes, one of which (a “quencher” or “quenching moiety”) is capable of quenching the fluorescence of the other dye (a “reporter” or “reporter moiety”). The dyes are attached to the probe, typically with the reporter or detector dye attached to the 5′ terminus and the quenching dye attached to an internal site, such that quenching occurs when the probe is in an unhybridized state and such that cleavage of the probe by the 5′ to 3′ exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673 describe alternative methods for detecting the degradation of probe that occurs concomitant with amplification.

A 5′ nuclease assay for the detection of a target nucleic acid can employ any polymerase that has a 5′ to 3′ exonuclease activity. In some embodiments, the polymerases with 5′-nuclease activity are thermostable and thermoactive nucleic acid polymerases. Such thermostable polymerases include, but are not limited to, native and recombinant forms of polymerases from a variety of species of the eubacterial genera Thermus, Thermatoga, and Thermosipho, as well as chimeric forms thereof.

For example, Thermus species polymerases that can be used in the methods of the invention include Thermus aquaticus (Taq) DNA polymerase, Thermus thermophilus (Tth) DNA polymerase, Thermus species Z05 (Z05) DNA polymerase, and Thermus species sps17 (sps17) DNA polymerase (e.g., described in U.S. Pat. Nos. 5,405,774; 5,352,600; 5,079,352; 4,889,818; 5,466,591; 5,618,711; 5,674,738, and 5,795,762). Thermatoga polymerases that can be used in the methods of the invention include, for example, Thermatoga maritima DNA polymerase and Thermatoga neapolitana DNA polymerase, while an example of a Thermosipho polymerase that can be used is Thermosipho africanus DNA polymerase. The sequences of Thermatoga maritima and Thermosipho africanus DNA polymerases are published in International Patent Application No. PCT/US91/07035 with Publication No. WO 92/06200. The sequence of Thermatoga neapolitana may be found in International Patent Publication No. WO 97/09451.

In the 5′ nuclease assay, the amplification detection is typically concurrent with amplification (i.e., “real-time”). In some embodiments, the amplification detection is quantitative, and the amplification detection is real-time. In some embodiments, the amplification detection is qualitative (e.g., end-point detection of the presence or absence of a target nucleic acid). In some embodiments, the amplification detection is subsequent to amplification. In some embodiments, the amplification detection is qualitative, and the amplification detection is subsequent to amplification.

The term “tagged probe” or “tagged oligonucleotide probe” refers to an oligonucleotide probe that is based on an DNA probe architecture that allows discrimination of multiple targets in the same optical channel by measuring the fluorescence at different temperatures and relates with the “TAGS (Temperature assisted generation of signal)” technology as disclosed in U.S. Patent Publication No. 2018/0073064 and incorporated by reference herein in its entirety. Multiplex PCR assays using only one reporter moiety (e.g. one fluorescent dye) is possible by designing tagged probes that have tag portions hybridized to their respective quenching oligonucleotide molecules at various melting temperatures (Tm). A first tagged probe with a low Tm tag-quenching oligonucleotide duplex may be used to detect a first target nucleic acid by measuring the calculated fluorescent value at a first temperature at or above its Tm temperature. A second tagged probe with a high Tm tag-quenching oligonucleotide duplex may be used to detect a second target by measuring the calculated fluorescent value at a second temperature at or above its Tm temperature and that is higher than the first temperature. As such, each optical channel for a given dye can be read in different “thermal channels” which represent the fluorescence measurements at different temperatures.

Stokes Shift and Large Stokes Shift Dyes

The Stokes shift of a fluorescent dye is defined as the wavelength, frequency, or energy difference between the absorption and emission peak maxima for the same electronic transition. The vast majority of small-molecule fluorophores exhibit Stokes shifts in the order of 10-25 nm and typically less than 80 nm (referred herein as “small Stokes shift” (SSS) dyes). SSS dyes include the conventional fluorescent dyes used in PCR assays, such as FAM, HEX, CFR610, and Quasar670. Fluorophores with significantly larger Stokes shifts are loosely referred to as “large Stokes shift” (LSS) dyes, “high Stokes shift” dyes, or “MegaStokes” dyes. The terminology is not well defined, but dyes with Stokes shifts >80 nm are typically coined with the adjective “large” or “high”, whereas the term “Mega” appears to be prevalently used for dyes with Stokes shifts significantly beyond 100 nm. Two photophysical mechanisms are discussed in the literature to explain the occurrence of large Stokes shifts. The molecular geometry-type mechanism is based on the conformational relaxation of the fluorophore in the excited state and the resulting rearrangement of the solvent dipoles. The Stokes shift grows with the increasing difference between the (equilibrated) molecular geometries and dipole moments in the ground and excited states. Large Stokes shift fluorescence for the electronic-type mechanism is ascribed to intramolecular charge transfer (ICT) in the excited state.

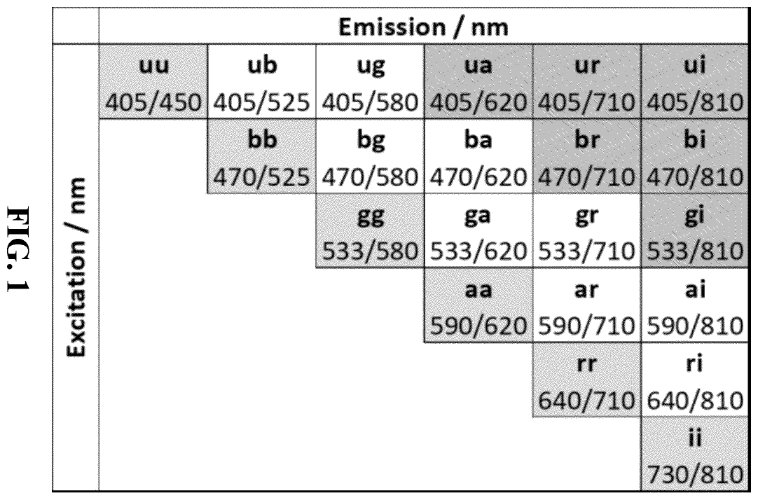

A common problem of fluorophores with small Stokes shift is internal quenching of fluorescence. Such self-quenching is caused by spectral overlap of excitation and emission, and especially prevalent at high fluorophore concentrations. LSS dyes have better separated spectral bands, which minimizes the reabsorption of photons. There is a non-zero probability for excitation of fluorophores outside of their major excitation peak. In consequence, fluorescence from one dye inevitably contributes to the total light detected in multiple emission channels. This spectral “cross-talk” or “bleed-through” can, to some extent, be compensated for computationally, by using predetermined correction factors. Additionally, scattering of excitation light adds to the background fluorescence in neighboring channels. LSS dyes allow to reduce or even avoid cross talk and scattering from other fluorophores. LSS dyes are especially useful in experimental settings where many fluorophores generate a strong background signal. Large spectral separation as for LSS dyes allows for more effective filtering of the excitation light, thereby enhancing the sensitivity of target detection. LSS dyes give access to fluorescence data from previously inaccessible optical channels. Facilitated by broad spectral separation, and when used in combination with standard fluorophores, LSS dyes allow increasing the multiplexing capabilities of fluorometric PCR devices. This way LSS labels allow the implementation of additional channels to established four- to six-color instruments. In principle, 21 channels can be composed from the filter combinations of a six-color instrument (FIG. 1). However, in practice the number of channels is limited by the commercial availability of LSS dyes with sufficiently large Stokes shift. Based on a Stokes shift of 150 nm for LSS dyes that are currently available on the market, nine additional channels can be implemented (FIG. 1, white channels). The channels for standard dyes are highlighted in light grey, whereas dark grey indicates channels for which suitable LSS dyes are currently not available. Instead, resonance electron transfer (RET) probes produce large “virtual” Stokes shift and can also be used to access these channels.

Examples of commercially available LSS dyes include but are not limited to the following: ALEXA FLUOR 430, ATTO 430LS, ATTO 490LS, ATTO 390LS, CASCADE YELLOW, CF350, CHROMEO 494, CYTO 500 LSS, CYTO 510 LSS, CYTO 514 LSS, CYTO 520 LSS, DAPOXYL, DY 480XL, DY 481XL, DY 485XL, DY 510XL, DY 511XL, DY 520XL, DY 521XL, DY 601XL, DY 350XL, DY 360XL, DY 370XL, DY 375XL, DY 380XL, DY 395XL, DY 396XL, DYLIGHT 515-LS, DYLIGHT 485-LS, DYLIGHT 510-LS, DYLIGHT 521-LS, FURA 2, INDO 1, KROME ORANGE, LUB 04, LUCIFER YELLOW, NBD X, NILE RED, PULSAR 650, PYMPO, STAR 440SXP, STAR 470SXP, STAR 520SXP, VIOGREEN, CF 350, SETAU 405, and PACIFIC ORANGE.

Despite the described benefits, increased Stokes shift comes at a price; LSS dyes have lower fluorescence quantum yields compared to standard fluorophores. With the brightness of a fluorophore being defined as the multiplication product of the molar extinction coefficient and fluorescence quantum yield, LSS dyes are also less bright. Another aspect is that the 40-50 nm peak widths of standard fluorophores can double or triple for LSS dyes. Nevertheless, for multiplexing purposes, the superior spectral separation of LSS dyes outweighs reduced brightness and larger peak widths.

Cobas® Liat, a Multi-Segment Tubule PCR System

The present disclosure also describes multi-segment tubule PCR devices, consumables, and methods for processing samples using such devices and consumables. An example of such a system is the Cobas® Liat® PCR System (Roche Molecular Systems, Pleasanton, CA). The Cobas® Liat® System is comprised of the Liat® tube and Liat® analyzer (instrument). The assay utilizes a single-use disposable Liat® tube that holds the sample preparation and real-time PCR reagents, and facilitates the sample preparation and real-time PCR processes. The Liat® tube contains all required unit dose reagents pre-packed in tube segments, separated by frangible seals, in the order of reagent use.

The Liat® analyzer automates and integrates sample preparation, nucleic acid amplification, detection and quantitation of the target sequence in biological samples. The Liat® analyzer performs all assay steps from clinical sample and reports assay result automatically. During the testing process, multiple sample processing actuators of the analyzer compress the Liat® tube to selectively release reagents from tube segments, move the sample from one segment to another, and control reaction volume, temperature, and time to conduct sample preparation, nucleic acid extraction, target enrichment, inhibitor removal, nucleic acid elution and real-time PCR. An embedded microprocessor controls and coordinates the actions of these actuators to perform all required assay processes within the closed Liat® tube. To run the assay, a user loads sample into a Liat® tube and places the loaded Liat® tube into a Liat® analyzer. The analyzer will perform sample preparation, real-time PCR, result calculation and report. All the processes are controlled by the assay script.

The part of the assay script that controls the thermocycling profile is shown in Table 1 below. In this embodiment, fluorescence readings from the FAM label were taken at 58° C. and at a high temperature for each cycle beginning from cycle #6. One of skill in the art would recognize that the parameters described in Table 1 may be changed as necessary, e.g., temperatures, durations, and number of cycles all may be altered as needed.

TABLE 1

| | Setting | | |
|---|---|---|---|
| | Temperature | Time | Cycle |
| Steps | (° C.) | (seconds) | (number) |
| RT | 55 | 30 | 1 |
| | 60 | 60 | |
| | 65 | 115 | |
| PCR-1 | 95 | 5 | 5 |
| | 55 | 5 | |
| | 58 | 5 | |
| | 60 | 5 | |
| PCR-2 | 94 | 4 | 40 |
| | 58 | 4 | |
| | 58 | 12+ 0.086/cycle | |

In several embodiments, segmented tubules provide a convenient vessel for receiving, storing, processing, and/or analyzing a biological sample. In certain embodiments, the segmented tubule facilitates sample processing protocols involving multiple processing steps. In certain embodiments, a sample may be collected in a sample tubule and the tubule then positioned in an analyzer; the analyzer may then manipulate the tubule and its contents to process the sample. In one embodiment, a flexible tubule may be segmented into compartments by breakable seals. The individual segments may contain various reagents and buffers for processing a sample. Clamps and actuators in an analyzer may apply, hold, and/or release force to the tubule in various combinations and with various timings to direct the movement of fluid and to cause the breakable seals to burst. This bursting of the breakable seals may create an inner tubule surface that is substantially free of obstructions to fluid flow. In some embodiments, the flow of the biological sample may be directed toward the distal end of the tubule as the processing progresses, while the flow of waste may be forced to move in the opposite direction, toward the opening of the tubule where the sample was initially input. This sample inlet can be sealed, optionally permanently, by a cap with a locking mechanism, and a waste chamber may be located in the cap to receive the waste for storage. A significant benefit of this approach is that the processed sample does not come into contact with surfaces that have been touched by the unprocessed sample. Consequently, trace amounts of reaction inhibitors present in the unprocessed sample that might coat the walls of the tubule are less likely to contaminate the processed sample.

In some embodiments, the tubule may be so expandable as to be capable of receiving a volume of fluid from each of multiple segments in one segment; this can allow sample and reagents to undergo certain processing steps in one segment leading to a simpler mechanical structure for performing assays. Another benefit of an embodiment using a tubule that may be so expandable is that the same tubule structure may be used to package different volumes of reagents within segments, allowing the same tubule to be packaged in differing ways depending upon the assay to be performed.

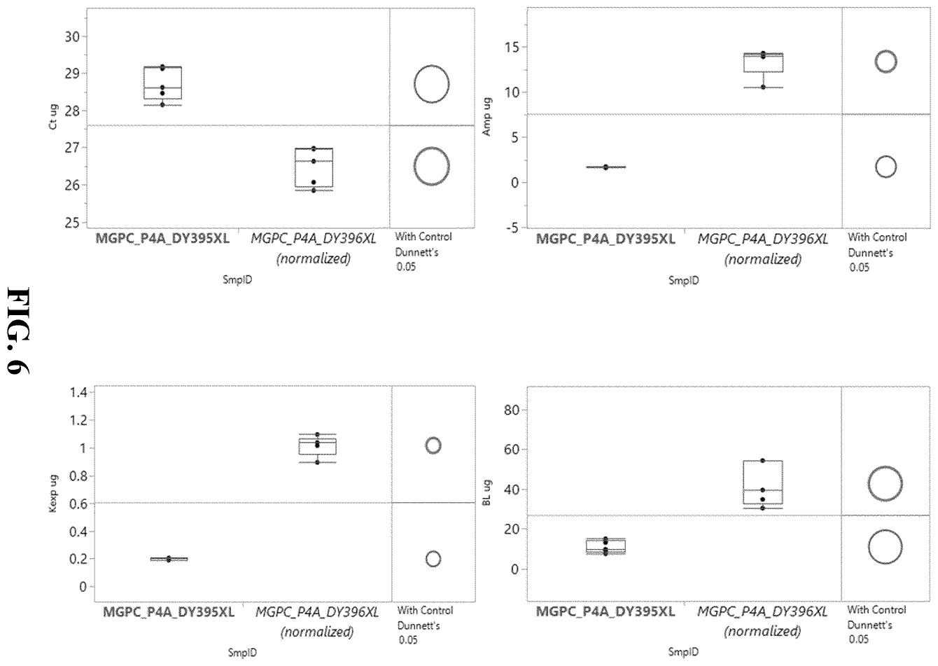

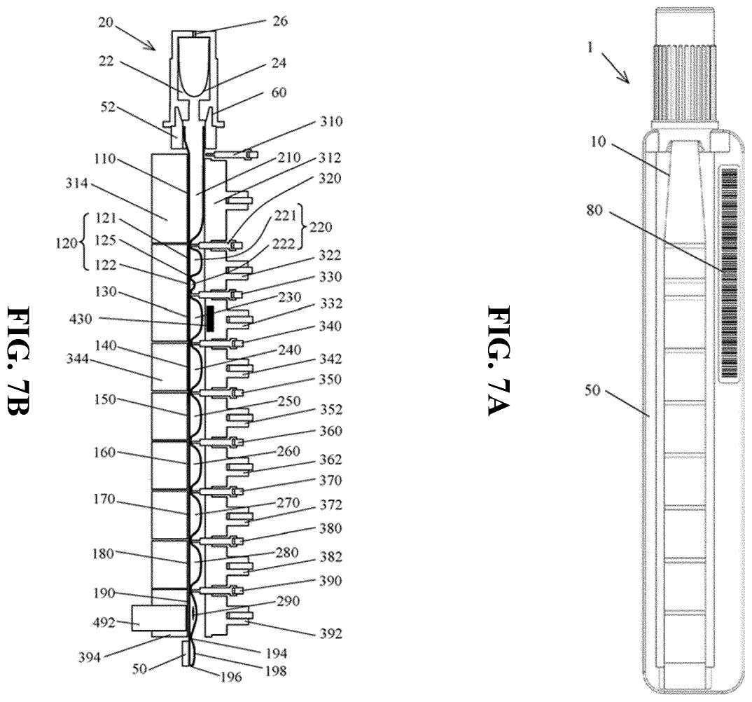

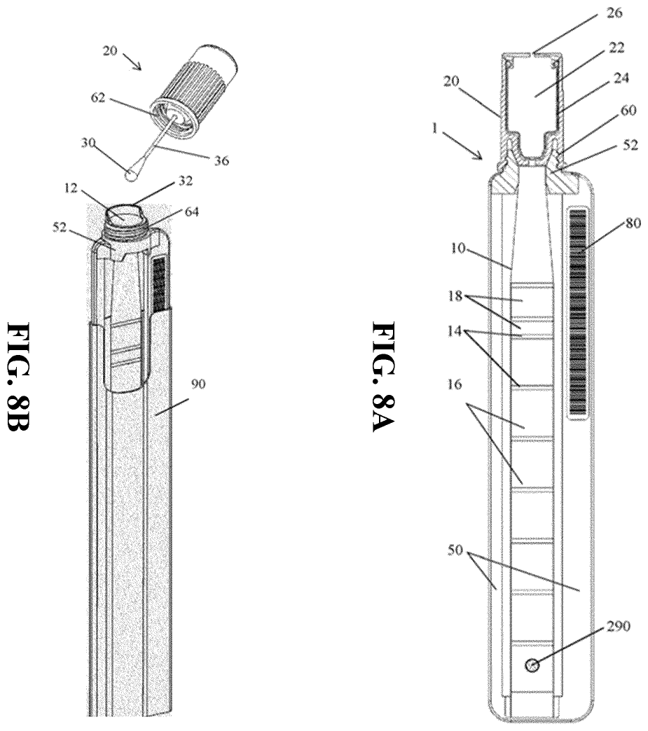

Referring to FIGS. 7A-B and FIGS. 8A-B, a transparent flexible tubule 10 is capable of being configured into a plurality of segments, such as 16, 110, 120, 130, 140, 150, 160, 170, 180, and/or 190, and being substantially flattened by compression. In an embodiment, a tubule may have at least two segments. The flexible tubule can provide operational functionality between approximately 2° C. and 105° C., compatibility with samples, targets and reagents, low gas permeability, minimal fluorescence properties, and/or resilience during repeated compression and flexure cycles. The tubule may be made of a variety of materials, examples of which include but are not limited to polyolefins such as polypropylene or polyethylene, polyurethane, polyolefin co-polymers and/or other materials providing suitable characteristics. The tubule properties, such as transparency, wetting properties, surface smoothness, surface charge and thermal resilience, may affect the performance of the tubule. These properties may be improved through such exemplary processes as seeding, plasma treating, addition of additives, and irradiation. In some embodiments, an additive material may be added to the plastic to improve selected characteristics. For example, a slip additive may be added, such as erucamide and/or oleamide; in some embodiment, a so-called “anti-block” additive may be added. An additive may have a concentration in the plastic in the range from about 0.01% to about 5.0%.

The tubule may be manufactured by a wide variety of suitable methods such as extrusion, injection molding and blow molding. In one embodiment, the tubule is continuously extruded. Alternative techniques for manufacturing the tubule include, e.g., casting, extruding or blowing films that can be fashioned by secondary processing operations into a suitable tubule. The tubule wall material may include multiple layers by co-extrusion, or by film lamination. For example, an inner layer may be chosen for high biocompatibility and an exterior layer may be chosen for low gas permeability. As a further example, the interior layer may be readily formed into a breakable seal 14 (FIG. 8A-B), such as a peelable seal, while the exterior layer may be resilient and highly impermeable. For example, the tubule may have a wall thickness of about 0.03 mm to about 0.8 mm, preferably 0.03 mm to about 0.5 mm, with the tubule able to be substantially flattened with an applied exterior pressure approximately one atmosphere.

Referring to FIG. 8A, in certain embodiments, the segments of the sample tubule 10 are defined by breakable seals 14 to fluidly isolate adjacent segments. This seal feature can be useful in separating, for example, a dry reagent from a liquid reagent until the two can be reconstituted to perform a specific assay, or for separating chemically reactive species until the reaction is desired. A breakable seal 14 may be formed in a region of the tubule 10 where opposing walls have been substantially joined, but not joined so strongly as to prevent the walls from being later peeled apart without significantly marring the tubule or the previously sealed surfaces. Such a seal may be termed a “peelable” seal. In some embodiments, the peelable seal region may be a band orthogonal to the axis of the tubule. It may span a tubule length in the range of about 0.5 mm to 5 mm, or about 1 mm to about 3 mm, most preferably about 1 mm. The seal preferably spans the entire width of the tubule so as to seal the segment. In some embodiments, the seal band may vary in height or shape and/or be oriented at an angle transverse to the axis of the tubule; such variations can change the peel characteristics.

Breakable seals 14, in the form of peelable seals, can be created between opposing walls of the tubule by applying a controlled amount of energy to the tubule in the location where the peelable seal is desired. For example, a temperature controlled sealing head can press the tubule at a specific pressure against a fixed anvil for a specific time interval. Various combinations of temperature, pressure and time may be selected to form a seal of desired size and peel strength. Energy may be delivered, for example, by a temperature controlled sealing head maintained at a constant temperature between 105° C. and 140° C. to heat a polypropylene tubing material; an actuator capable of delivering a precise pressure between 3 and 100 atmospheres over the desired seal region; and a control system to drive the sequencing of the actuator to a specific cycle time between 1 and 30 seconds. Using this method, satisfactory seals have been created in polypropylene tubules to peel open when subjected to an internal pressure approximately 1 atmosphere. Alternate techniques to deliver the sealing energy to the tubule include RF and ultrasonic welding.

In other embodiments, alternate tubule materials and blends of materials can be used to optimize peelable seal performance. For example, two polypropylene polymers of differing melting temperature can be blended in a ratio such that the composition and melt characteristics are optimized for peelable seal formation. Referring to FIG. 7B, in addition to or in lieu of breakable seals 14, the flexible tubule can further have one or more pressure gates 194, which are capable of reversibly opening and closing during the operation of a test by applying a controlled force to a segment of the flexible tubule.

A filter can be embedded in a tubule segment. In a preferred embodiment, a filter can be formed by stacking multiple layers of flexible filter material. The uppermost layer of the filter that directly contacts a sample may have a pore size selected for filtration; the bottom layer of the filter may include a material with much larger pore size to provide a support structure for the uppermost layer when a pressure is applied during filtration. In this preferred embodiment, the filter may be folded to form a bag, with the edges of its open end firmly attached to the tubule wall. The segment with the filter bag may be capable of being substantially flattened by compressing the exterior of the tubule.

In exemplary embodiments, one or more reagents can be stored either as dry substance and/or as liquid solutions in tubule segments. In embodiments where reagents may be stored in dry format, liquid solutions can be stored in adjoining segments to facilitate the reconstitution of the reagent solution. Examples of typical reagents include lysis reagent, elution buffer, wash buffer, DNase inhibitor, RNase inhibitor, proteinase inhibitor, chelating agent, neutralizing reagent, chaotropic salt solution, detergent, surfactant, anticoagulant, germinant solution, isopropanol, ethanol solution, antibody, nucleic acid probes, peptide nucleic acid probes, and phosphothioate nucleic acid probes. In embodiments where one of the reagents is a chaotropic salt solution, a preferred component is guanidinium isocyanate or guanidinium hydrochloride or a combination thereof. In some embodiments, the order in which reagents may be stored in the tubule relative to the opening through which a sample is input reflects the order in which the reagents can be used in methods utilizing the tube. In some embodiments, a reagent includes a substance capable of specific binding to a preselected component of a sample. For example, a substance may specifically bind to nucleic acid, or a nucleic acid probe may specifically bind to nucleic acids having particular base sequences.

In other embodiments, a solid phase substrate can be contained within a tubule segment and used to capture one or more selected components of a sample (if such component is present in a sample), such as a target microorganism or nucleic acids. Capturing can help to enrich the target component and to remove reaction inhibitors from a sample. Substrates may be solid phase materials that can capture target cells, virions, nucleic acids, or other selected components under defined chemical and temperature conditions, and may release the components under different chemical and temperature conditions.

In some embodiments, a reagent can be coated on the substrate. Examples of coatable reagents are receptors, ligands, antibodies, antigens, nucleic acid probes, peptide nucleic acid probes, phosphothioate nucleic acid probes, bacteriophages, silica, chaotropic salts, proteinases, DNases, RNases, DNase inhibitors, RNase inhibitors, and germinant solutions. In some embodiments, the substrate can be stored in a dry segment of the tubule while in other embodiments it can be stored immersed in a liquid. In some embodiments, the order in which reagents may be stored in the tubule relative to the substrate and the opening through which a sample is input, reflects the order in which the reagents and the substrate can be used in methods utilizing the apparatus.

The substrate can be: beads, pads, filters, sheets, and/or a portion of tubule wall surface or a collection tool. In embodiments where the substrate is a plurality of beads, the beads can be: silica beads, magnetic beads, silica magnetic beads, glass beads, nitrocellulose colloid beads, and magnetized nitrocellulose colloid beads. In some embodiments where the beads can be paramagnetic, the beads can be captured by a magnetic field. Examples of reagents that may permit the selective adsorption of nucleic acid molecules to a functional group-coated surface are described, for example, in U.S. Pat. Nos. 5,705,628; 5,898,071; and 6,534,262. Separation can be accomplished by manipulating the ionic strength and polyalkylene glycol concentration of the solution to selectively precipitate, and reversibly adsorb, the nucleic acids to a solid phase surface. When these solid phase surfaces are paramagnetic microparticles, the magnetic beads, to which the target nucleic acid molecules have been adsorbed, can be washed under conditions that retain the nucleic acids but not other molecules. The nucleic acid molecules isolated through this process are suitable for: capillary electrophoresis, nucleotide sequencing, reverse transcription, cloning, transfection, transduction, microinjection of mammalian cells, gene therapy protocols, the in vitro synthesis of RNA probes, cDNA library construction, and the polymerase chain reaction (PCR) amplification. Several companies offer magnetic-based purification systems, such as QIAGEN's MagAttract™, Cortex Biochem's MagaZorb™, Roche Applied Science's MagNA Pure LC™, and MagPrep® Silica from Merck & Co. All of these products use negatively charged particles and manipulate buffer conditions to selectively bind a variety of nucleic acids to the beads, wash the beads and elute the beads in aqueous buffers. Many of the products used by these companies use chaotropic salts to aid in the precipitation of nucleic acids onto the magnetic beads. Examples are described in U.S. Pat. Nos. 4,427,580; 4,483,920; and 5,234,809.

In some embodiments, the substrate may be a pad. In further embodiments, the substrate pad can include paper, alternating layers of papers with different hydrophobic properties, glass fiber filters, or polycarbonate filters with defined pore sizes. In some embodiments, the pad may be a filter or impermeable sheet for covering selected portion of the surfaces of the pad, the filter having a predetermined pore size. Such a filtration device can be used for separations of white blood cells and red blood cells (or other particles, such as virus or microorganisms) from whole blood and/or other samples. The pad can be mounted on the tubule wall and/or on a sample collection tool. In some embodiments, the pad can be soaked with a reagent solution while in other embodiments it may be coated with dry reagents.

Preferred exemplary embodiments may include a linear arrangement of 2 or more tubule segments 110, 120, 130, 140, 150, 160, 170, 180, and/or 190 (FIG. 7B). A linear arrangement facilitates moving the sample and resultant waste and target through the tube in a controlled manner. A raw biological sample can be input through a first opening 12 (FIG. 8B) in a first segment 110 (FIG. 7B) of the tubule. Thereafter, waste from a processed sample can be moved back toward the first opening while the target is pushed towards the opposite end, thereby minimizing contamination of the target by reaction inhibitors that may have become attached to the tubule wall, and confining the target to a clean segment of the tubule which can contain suitable reagents for further operations of the target. Some embodiments may use a plurality of at least three segments, each containing at least one reagent. In some embodiments, these segments may contain reagents in the following order: the reagent in the second segment may be either a lysis reagent, a dilution or wash buffer, or a substrate; the reagent in the third segment may be either a substrate, a lysis reagent, a washing buffer or a neutralization reagent; the reagent in the fourth segment may be a wash buffer, a suspension buffer, an elution reagent, or nucleic acid amplification and detection reagents. In some embodiments, the three segments may be arranged continuously, while in other embodiments, these three segments may be separated by another segment or segments in between via breakable seals.

In some embodiments, a pressure gate 194 (FIG. 7B) can be incorporated to selectively close and open a second opening, located at the distal end of the tubule, to collect the products generated during a test from the tubule for further processing, outside of the tubule. In some embodiments, this second opening may located in a segment 198 defined by two pressure gates 194 and 196 to store a product from the sample processing segments. In some embodiments, a combination of a breakable seal and a pressure gate may be provided for transferring the contents of the tubule to a second opening.

In some embodiments a tube closing device for closing the tube after sample input may include a cap 20 (FIG. 7B) and/or clamp 310. An interface or adaptor 52 between the cap and the first opening of the flexible tubule may be used to ensure a secure, hermetic seal. In an exemplary embodiment, this interface may be threaded and may include tapered features 62 on the cap and/or a suitably rigid tube frame 50 such that, when fastened together, the threads 64 can engage to mate the tapered features 62 between the tube frame and cap to provide a suitable lock. In this exemplary embodiment, the cap may require ½ to 1 full rotation to fully remove or attach from the tube holder. The combination of thread pitch and taper angle in the joint can be selected to be both easily manufactured and to provide feedback resistance to inform the user that an effective seal has been created. In other embodiments the cap locking device may include snap fits, press fits, and/or other types of “twist and lock” mechanism between the cap and tube holder, and similar arrangements in which the cap is permanently attached to the tubule, such as by hinging or tethering the cap.

Both the cap 20 and tube frame 50 can be made of a suitable injection molded plastic such as polypropylene. The tube frame 50 can, in turn, be fastened to the flexible tube by a permanent, hermetic seal. The exterior portion of the cap may be covered with ridges or finger grips to facilitate its handling. Furthermore, the cap 20 may include an area for attaching a sample identification mark or label. As a further alternative, the cap may be directly attached to the first opening flexible tube through a press fit or a collar that compresses the flexible tube opening against a protrusion in the cap to create a hermetic seal. The lock between the tube cap and tube holder may be keyed or guided such that a collection tool 36 or features integrated into the cap can be definitively oriented with respect to the tube to facilitate sample processing and the flattening of the flexible tubule. Furthermore, the cap may incorporate features such as a ratchet or similar safety mechanism to prevent the cap from being removed after it has been installed onto the opening of the flexible tube.

The cap 20 used to close the tubule in some embodiments may contain a cavity 22 within it by making the cap body substantially hollow. In some embodiments, the hollow portion extends from the top of the cap body to an orifice at the base of the cap body. To form a chamber, the top of the cavity may be closed by fastening a cover onto the cap body. The cover may be constructed of the same piece as the cap body. The cover may incorporate a vent hole 26 or may further incorporate an affixed microbe barrier, filter or a material that expands to close off the vent hole when exposed to a liquid or specific emperature. The bottom of the chamber may be left open or closed by a breakable septum or valve. The hollow chamber may further incorporate a flexible membrane or septum 24. This flexible septum could be manufactured using dip molding, liquid injection silicone molding, blow molding, and/or other methods suitable for the creation of thin elastomeric structures. The flexible septum can be inserted into the cap body cavity 22 assembly so as to effectively isolate the interior portion of the tube from the exterior environment after the cap is in place on the tube. The flexible septum could be designed such that, in the absence of externally applied pressures, its inherent stiffness ensures it is in a preferred, known state of deformation. As a further embodiment, the flexible septum may be replaced by a plunger. In an exemplary embodiment, a cap body approximately 30 mm high by 14 mm diameter may be injection molded of a suitable thermoplastic and contain an interior cavity having at least 500 uL of available volume. The chamber in the cap body could be adapted for useful purposes such as holding or dispensing a reagent, serving as a reservoir to hold waste fluids, serving as a retraction space for an integrated collection tool, or a combination of thereof.

The cap 20 may have an integrated collection tool 30 (FIG. 8B) such as a swab, capillary tube, liquid dropper, inoculation loop, syringe, absorbent pad, forceps, scoop or stick to facilitate the collection of liquid and solid samples and their insertion into the tubule. The collection tool may be designed to collect and deposit a predetermined amount of material into the tube. Reagents may be stored on the collection tool itself. For example, the collection tool may include a swab impregnated with a dry salt such that when the swab is hydrated it would suspend the salt off the swab into solution. Furthermore, the collection tool and cap may be designed such that the collection tool portion retracts into the cap body after depositing the sample into the tubule to leave the tubule segments substantially unencumbered.

The chamber 22 in the cap 20 may be fashioned to store a reagent. To accomplish this, for example, the base of the chamber may be closed by a breakable septum or valve (not shown) such that when the cap is squeezed, the septum breaks to release the reagent. Such a feature would be useful, for example, if the cap were integrally formed with a collection tool such as a swab or stick. In this instance, the reagent released from the cap chamber could be used to wash a sample off the collection tool into a tube segment or to lyse the sample contained on the collection tool. Reagents may also be released from the cap chamber by opening the breakable septum using pressure generated by compressing a flexible tube segment to force fluid from the tube up into the cap chamber. The chamber in the cap may be fashioned to store waste fluids derived from processing within the tubule. In another embodiment, the base of the chamber may be left open such that when connected to the first opening of the flexible tubule a fluid passage is formed between the tubule and the chamber. As fluid is moved into the cap chamber, the flexible septum 24 contained within can move from an initial position upward so as to accommodate the influx of new fluid. This septum movement can be facilitated by the incorporation of a vent hole 26 on the cap body cover. Referring to FIG. 7B, after fluid has been transferred into the cap chamber a clamp 310 or actuator 312 in the analyzer can act to compress the tubule and effectively seal off the cap chamber volume from the tubule segments. As an alternative embodiment, the cap chamber may incorporate a pressure gate or check valve (not shown) to prohibit fluid flow from the cap chamber back into the tube segments. As a further alternative, the flexible septum may be omitted with the cap chamber cover including a microbe barrier to permit the free escape of contained gasses but retain all the liquid volumes and infectious agents in the tube. As a further alternative, the flexible septum can be replaced with a plunger that would move axially upward to accommodate additional fluid volumes transferred from the tube segments to the cap chamber. Other methods to accommodate fluidic waste within the cap chamber can be readily envisioned without departing from the scope of the present disclosure.

A substantially rigid frame 50 (FIG. 7A) may be provided to hold the flexible tubule 10 suitably taut by constraining at least the proximal and distal ends of the tubule. In an exemplary embodiment, a first constraint may be provided to permanently attach and seal the tubule to the frame around the first opening of the tube. This seal may be created by welding the flexible tubule to the frame using thermal and/or ultrasonic sources. Alternatively, the seal may be created using a hot-melt adhesive joint with ethylene vinyl acetate, or by making a joint using a UV cure epoxy or other adhesives. In further embodiments, the tubule may be mechanically sealed or insert-molded with the frame. A second constraint may be provided to attach and seal the tubule to the base of the frame. In an exemplary embodiment of this second constraint, this end of the tubule may be sealed flat and attached to the rigid frame by thermal and/or ultrasonic welding techniques. Alternatively, this joint and seal may also be formed using adhesive or mechanical approaches. In an alternative embodiment, the second seal may be similar to the first seal, being substantially open to enable access to the contents of the flexible tubule from the second opening. The tubule and frame materials can be optimized for joint manufacture. For example, the frame can be made of polypropylene having a lower melting point than the thinner tubule to ensure more uniform melting across one or more weld zones. To facilitate welding between the tubule and the frame, the joint area may be tapered or otherwise shaped to include energy directors or other commonly used features enhance weld performance. In an exemplary embodiment, the rigid frame can be made of any suitable plastic by injection molding with its dimensions being approximately 150 mm tall by 25 mm wide.

The rigid frame 50 can incorporate several features to facilitate the compression and flattening of the flexible tubule. For example, in an exemplary embodiment, the flexible tubule 10 may be constrained only at its two axial extremities to allow maximum radial freedom to avoid encumbering the tubule's radial movement as it is compressed. In another embodiment, compression may be facilitated by including a relief area in the frame, near the first opening of the tube. This relief area may be used to facilitate the flexible tubule's transition from a substantially compressed shape in the tubule segments to a substantially open shape at the first opening. Other useful features of the rigid frame that can facilitate flexible tubule compression may include an integral tubule tensioning mechanism. In an exemplary embodiment, this tension mechanism could be manufactured by molding features such as cantilever or leaf type springs directly into the rigid frame to pull the tubule taut at one of its attachment points with the frame.

The rigid frame 50 can facilitate tube identification, handling, sample loading and interfacing to the tube cap. For example, the frame can provide additional area to identify the tube through labels or writing 80 affixed thereto. The plastic materials of the frame may be color coded with the cap materials to help identify the apparatus and its function. The frame may incorporate special features such as changes in thickness or keys to guide its orientation into a receiving instrument or during manufacture. The frame may interface to a sleeve 90 or packaging that covers or protects the flexible tubule from accidental handling damage, light exposure, and/or heat exposure. The body of the rigid frame may also provide a convenient structure to hold the tube. The frame may have an integral collection tool 32 such as a deflector or scoop to facilitate sample collection into the apparatus. The sample-receiving end of the frame may also incorporate a tapered or funneled interior surface to guide collected sample into the flexible tube.

In some embodiments, a method of extracting nucleic acids from biological samples by using the apparatus described in the previous paragraphs is contemplated. In certain embodiments, the sequence of events in such a test may include: 1) a biological sample can be collected with a collection tool, 2) the collected sample can be placed into a flexible tubule, which can include a plurality of segments that may contain the reagents required during the test, through a first opening in the tubule, 3) at least one substrate may be set at a controlled temperature and/or other conditions to capture target organisms or nucleic acids during a set incubation period, 4) organisms or molecules, in the unprocessed sample, that may not bind to the substrate can thus be removed by transferring liquid to a waste reservoir, 5) waste may be stored in a waste reservoir, that can be segregated from the target by a clamp and/or actuator compressed against the tubule, 6) a wash buffer, released from another segment of the tubule, may be added to remove reaction inhibitors, 7) an elution reagent, from another segment, may be added to release the target bound to the substrate after incubation at a controlled temperature, and 8) nucleic acids can be detected by techniques well known to those familiar in the art or collected through a second opening in the tubule. In exemplary embodiments, the flow of the sample may be from the first opening towards the distal end of the tubule as the test progresses while the flow of waste may be towards the closed sample input opening of the tubule, where a waste chamber in the cap of the tubule receives the waste for storage. Consequently, undesirable contact between a processed sample and surfaces in a reaction vessel that have been touched by the unprocessed sample is avoided, thereby preventing reaction inhibition due to trace amounts of reaction inhibitors present in the unprocessed sample and that might coat the walls of the reaction vessel.

Some embodiments may incorporate the use of a test tube 1 (FIGS. 7A-B), with a flexible tubule divided into a plurality of segments, such as segments 16, 110, 120, 130, 140, 150, 160, 170, 180, and/or 190, that may be transverse to the longitudinal axis of the tubule, and which may contain reagents, such as reagents 210, 221, 222, 230, 240, 250, 260, 270, 280, and/or 290; as well as an analyzer, that may have a plurality of actuators, such as actuators 312, 322, 332, 342, 352, 362, 372, 382, and/or 392, clamps, such as clamps 310, 320, 330, 340, 350, 360, 370, 380, and/or 390, and blocks, for example 314, 344, and/or 394 (others unnumbered for simplicity); opposing the actuators and clamps, to process a sample. Various combinations of these actuators, clamps, and/or blocks may be used to effectively clamp the tubule closed thereby segregating fluid. In exemplary embodiments, at least one of the actuators or blocks may have a thermal control element to control the temperature of a tubule segment for sample processing. The sample processing apparatus can further have at least one magnetic field source 430 capable of applying a magnetic field to a segment. The sample processing apparatus can further have a detection device 492, such as photometer or a CCD, to monitor a reaction taking place or completed within the tubule.

The combined use of the tube and the analyzer can enable many sample-processing operations. Collecting a sample, such as blood, saliva, serum, soil, tissue biopsy, stool or other solid or liquid samples, can be accomplished by using a sample collection tool 30 that may be incorporated into the cap 20, or features 32 on the tube frame 50. After a suitable amount of the sample has been collected, the cap can be placed onto the first opening of the tube to close the tube and deposit the sample into the first segment. Following this step, the sample contained on the collection tool may be washed off or resuspended with reagents contained in separate chambers within the cap by compressing a portion of the cap. The tube can then be loaded into the analyzer for further processing. Identification features, such as a barcode or an RF tag, can be present on the tube to designate the sample's identity in a format that can be read by the analyzer and/or a user.

Opening a breakable seal of a tubule segment can be accomplished by applying pressure to the flexible tubule to irreversibly separate the bound surfaces of the tubule wall. An actuator can be used to apply the required pressure to compress a tubule segments containing fluid to open a breakable seal. In embodiments where a segment is delimited by two breakable seals, A and B, the analyzer may preferentially break seal A by physically protecting the seal B region with an actuator or clamp to prevent seal B from breaking while pressure is applied to the segment to break seal A. Alternatively, seal A may be preferentially opened by applying pressure to the segment adjacent to seal A in a precise manner such that; seal A is first opened by the pressure created in the adjacent segment; after seal A is broken, the pressure between the two segments drops substantially due to the additional, combined, segment volume; the reduced pressure in the combined segment is insufficient to break seal B. This method can be used to open breakable seals one at a time without using a protecting actuator and/or clamp. As a further alternative, the adherence of seal A may be inferior to that of seal B such that seal A can break at a lower pressure than seal B.

A process of moving fluid from one segment to another segment may include, for example, releasing a clamp on one end of the first segment, compressing a clamp on the other end of the first segment, releasing an actuator on the second segment, and compressing an actuator on the first segment to move the liquid from the first segment to the second segment. Alternatively, the clamp may be omitted or be opened after releasing the actuator on the second segment.

A process of mixing two substances, where at least one is liquid, located in adjacent segments may be accomplished by: releasing the clamp between the two segments, moving the liquid contained in the first segment, through an opened breakable seal to the second segment; and alternatively compressing the second segment and the first segment to flow the liquid between the segments.

An agitation can be performed by alternatively compressing and decompressing a tubule segment with an actuator, while both clamps that flank the actuator are compressing the tubule. In another embodiment, agitation can be achieved by alternatively moving liquid between at least two segments.

In embodiments where a tubule segment may contain a liquid having a volume exceeding the volume required for a protocol, a process of adjusting the volume of the liquid in the segment can be executed by: compressing the tubule segment to reduce the gap of between the tube walls to set the volume of the segment to a desired level and allowing the exceeding liquid to flow to the adjacent segment, past a clamp at the end of the segment or adjacent actuator; closing the tubule segment with the clamp or actuator, resulting in an adjusted volume of liquid remaining in the segment.

A process of removing air bubbles may include agitating a segment containing the bubbly liquid. Another process of removing air bubbles may include agitating a first segment containing liquid while closing a second segment; opening the second segment and moving the liquid from the first segment to the second segment; agitating the second segment and adjusting a position of the second actuator to move the liquid-air interface near or above the upper end of the second segment, then clamping the upper end of the second segment to form a fully liquid-infused segment without air bubbles.

A dilution process can be conducted by using the liquid movement process wherein one of the segments includes a diluent and the other includes a substance to be diluted.

A process of reconstituting a reagent from dry and liquid components separately stored in different tubule segments or sub-segments may include compressing the tubule segment or sub-segment containing the liquid components to open the breakable seal connecting to the dry reagent segment, moving the liquid into the dry reagent segment or sub-segment, and mixing the dry reagent and liquid components using the mixing process.

Incubation of the contents in a segment can be achieved by setting the corresponding actuator and/or block temperature and applying pressure to the segment to ensure a sufficient surface contact between the tubule wall of the segment and the actuator and the block, and bring the contents of the tubule segment to substantially the same temperature as the surrounding actuator and/or block temperature. The incubation can be conducted in all processing conditions as long as the temperatures of all involved segments are set as required.

Rapid temperature ramping for incubation can be achieved by incubating a fluid in a first segment at a first temperature and setting a second temperature for a second segment adjoining the first segment, after incubation at the first temperature is finished, liquid is rapidly moved from the first segment to the second segment and incubated at the second temperature.

A flow driving through a flow-channel process can be performed by compressing the tubule with a centrally positioned actuator, and its flanking clamps if any, to form a thin-layer flow channel with a gap of about 1 to about 500 μm, preferably about 5 to about 500 μm through segment. The adjacent actuators compress gently on the adjacent segments in liquid communication with the flow-channel to generate an offset inner pressure to ensure a substantially uniform gap of the thin-layer flow channel. The two flanking actuators can then alternatively compress and release pressure on the tubule on their respective segments to generate flow at controlled flow rate. Optional flow, pressure, and/or force sensors may be incorporated to enable closed-loop control of the flow behavior. The flow-channel process can be used in washing, enhancing the substrate binding efficiency, and detection.

A magnetic bead immobilization and re-suspension process can be used to separate the beads from the sample liquid. The magnetic field generated by a magnetic source 430 (FIG. 7B) may be applied to a segment 130 containing a magnetic bead suspension 230 to capture and immobilize the beads to the tube wall. An agitation process can be used during the capturing process. In another embodiment, a flow-channel can be formed on the segment with the applied magnetic field, and magnetic beads can be captured under flow to increase the capturing efficiency. For re-suspending immobilized beads, the magnetic field may be turned off or removed, and an agitation or flow-channel process can be used for resuspension.

A washing process to remove residual debris and reaction inhibitors from a substrate may be conducted by using three basic steps: First, an actuator can compress a segment containing the substrate, such as immobilized beads or a sheet, to substantially remove the liquid from this segment. Second, a washing buffer may be moved to the segment by using a process similar to that of reconstituting a reagent from dry and liquid components. For bead-based substrates, a bead re-suspension process can be used followed by bead re-capture on the tubule wall. Third, after a mixing or agitation process, the actuator can compress the segment to remove the used wash liquid from the segment. In another embodiment, a flow-channel can be formed in the segment containing a substrate, which may be either immobilized beads or a sheet. A unidirectional flow wash, having laminar characteristics, is generated through the flow channel with the substrate. Finally, all the actuators and clamps, if any, can be closed to remove substantially all the liquid from the segments. In a further embodiment, a combination of the dilution based washing and the laminar flow based washing can be used to further enhance the washing efficiency.

Lysis can be achieved by heating a sample at a set temperature or by using a combination of heat and chemical agents to break open cell membranes, cell walls or uncoated virus particles. In another embodiment, lysis can be achieved using a chemical reagent, such as proteinase K, and a chaotropic salt solution. The chemical reagents can be stored in one of more tubule segments and combined with the sample using the processes disclosed above. In some embodiments, multiple processes such as chemical cell lysis, mechanical grinding and heating, can be combined to break up solid sample, for example tissue collected from biopsy, to maximize the performance. Capturing target microorganisms can be achieved by using a substrate. In an embodiment, the surface of the substrate may be coated with at least one binding reagent, such as an antibody, ligand or receptor against an antigen, receptor or ligand on the surface of the target organism (ASA), a nucleic acid (NA), a peptide nucleic acid (PNA) and phosphothioate (PT) nucleic acid probe to capture a specific nucleic acid target sequence complementary to the probe or a target organism. In another embodiment, the surface may be selected to have, or coated to form, an electrostatically charged (EC) surface, such as silica- or ion exchange resin-coated surface, to reversibly capture substantially only nucleic acids. In some embodiments, the substrate may be pre-packed in a tubule segment or subsegment in dry format, and a liquid binding buffer may be packed in another segment. The substrate and the buffer can be reconstituted by using the aforementioned processes. In some embodiments, a reagent from an adjoining segment can be used to dilute the sample before incubation with the substrate. In some embodiments, the target organisms can be captured to the substrate prior to lysing the microorganisms; while in other embodiments, a lysis step can be conducted before the target-capturing step. In preferred embodiments, incubation of the substrate in agitation can be conducted at a desired temperature, for example, at 4° C. for live bacterial capture, or room temperature for viral capture. Capture can be followed by a washing process to remove the residues and unwanted components of the sample from the tubule segment.

In some embodiments, magnetic beads can be used as the substrate for capturing target, and a magnetic bead immobilization and re-suspension process may be used to separate the beads from the sample liquid. In other embodiments where the substrate may be a pad or a sheet, the substrate may be incorporated into the collection tool and/or may be adhered on the tubule wall in a segment. Elution can be achieved by heating and/or incubating the substrate in a solution in a tubule segment at an elevated temperature. Preferred temperatures for elution are from 50° C. to 95° C. In another embodiment, elution may be achieved by changing the pH of the solution in which the substrate is suspended or embedded. For example, in an exemplary embodiment the pH of the wash solution can be between 4 and 5.5 while that of the elution buffer can be between 8 and 9.

A spore germination process can be conducted by mixing a sample containing bacterial spores with germination solution, and incubating the mixture at a suitable condition. The germinant solution may contain at least one of L-alanine, inosine, L-phenylalanine, and/or L-proline as well as some rich growth media to allow for partial growth of the pre-vegetative cells released from the spores. Preferred incubation temperatures for germination range from 20° C. to 37° C. By coating the substrate with an anti-spore antibody, vegetative cells can be selectively enriched from a sample that contains both live and/or dead spores. The live spores can release a plurality of vegetative cells from the substrate, which can be further processed to detect nucleic acid sequences characteristic of the bacterial species. In some embodiments, the germinant solution can be absorbed in a pad.

In certain embodiments, nucleic acids extracted from the biological samples may be further processed by amplifying the nucleic acids using at least one method from the group consisting of: polymerase chain reaction (PCR), rolling circle amplification (RCA), ligase chain reaction (LCR), transcription mediated amplification (TMA), nucleic acid sequence based amplification (NASBA), and strand displacement amplification reaction (SDA). In some embodiments, the nucleic acids extracted from the organism can be ribonucleic acids (RNA) and their processing may include a coupled reverse transcription and polymerase chain reaction (RT-PCR) using combinations of enzymes such as Tth polymerase and Taq polymerase or reverse transcriptase and Taq polymerase. In some embodiments, nicked circular nucleic acid probes can be circularized using T4 DNA ligase or Ampligase™ and guide nucleic acids, such as DNA or RNA targets, followed by detecting the formation of the closed circularized probes after an in vitro selection process. Such detection can be through PCR, TMA, RCA, LCR, NASBA or SDAR using enzymes known to those familiar with the art. In exemplary embodiments, the amplification of the nucleic acids can be detected in real time by using fluorescent-labeled nucleic acid probes or DNA intercalating dyes as well as a photometer or charge-coupled device in the molecular analyzer to detect the increase in fluorescence during the nucleic acid amplification. These fluorescently-labeled probes use detection schemes well known to those familiar in the art (i.e., TaqMan™, molecular Beacons™, fluorescence resonance energy transfer (FRET) probes, Scorpion™ probes) and generally use fluorescence quenching as well as the release of quenching or fluorescence energy transfer from one reporter to another to detect the synthesis or presence of specific nucleic acids.

A real-time detection of a signal from a tubule segment can be achieved by using a sensor 492 (FIG. 7B), such as a photometer, a spectrometer, a CCD, connected to a block, such as block 490. In exemplary embodiments, pressure can be applied by an actuator 392 on the tubule segment 190 to suitably define the tubule segment's shape. The format of signal can be an intensity of a light at certain wavelength, such as a fluorescent light, a spectrum, and/or an image, such as image of cells or manmade elements such as quantum dots. For fluorescence detection, an excitation of light from the optical system can be used to illuminate a reaction, and emission light can be detected by the photometer. To detect a plurality of signals having specific wavelengths, different wavelength signals can be detected in series or parallel by dedicated detection channels or a spectrometer. The disclosed devices and methods can be widely applied in the practice of medicine, agriculture and environmental monitoring as well as many other biological sample-testing applications. Nucleic acids isolated from tissue biopsy samples that surround tumors removed by a surgeon can be used to detect pre-cancerous tissues. In these applications, hot-spot mutations in tumor suppressor genes and proto-oncogenes can be detected using genotyping techniques well known to those familiar with the art. Precancerous tissues often have somatic mutations that can readily be identified by comparing the outcome of the genotyping test with the biopsy sample to the patient's genotype using whole blood as a source of nucleic acids. Nucleic acids isolated from white blood can be used to detect genetic variants and germline mutations using genotyping techniques well known to those familiar with the art. Examples of such mutations are the approximately 25 known mutants of the CFTR gene recommended for prenatal diagnosis by the American College of Medical Genetics and the American College of Obstetricians and Gynecologists. Examples of genetic variants are high frequency alleles in glucose-6-phosphate dehydrogenase that influence sensitivity to therapeutic agents, like the antimalarial drug Primaquine.

Another example of genetic variations with clinical relevance are alleles pertaining to increased risks of pathological conditions, like the Factor V Leiden allele and the increased risk of venous thrombosis. Nucleic acids isolated from bacteria can be used to detect gene-coding sequences to evaluate the pathogenicity of a bacterial strain. Examples of such genes are the Lethal Factor, the Protective Antigen A, and the Edema factor genes on the PXO1 plasmid of Bacillus anthracis and the Capsular antigen A, B, and C on the PXO2 plasmid of B. anthracis. The presence of these sequences allows researchers to distinguish between B. anthracis and harmless soil bacteria. Nucleic acids isolated from RNA viruses can be used to detect gene-coding sequences to detect the presence or absence of a virus or to quantify a virus in order to guide therapeutic treatment of infected individuals.

A particularly significant utility of such assays is the detection of the human immunodeficiency virus (HIV), to guide anti-retroviral therapy. Nucleic acids isolated from DNA viruses can be used detect gene coding sequences to detect the presence or absence of a virus in blood prior to their use in the manufacturing of blood derived products. The detection of hepatitis B virus in pools of blood samples is a well-known example of this utility to those familiar in the art. The presence of verotoxin Escherichia coli in ground beef is a good example of the potential agricultural uses of the apparatus. Detecting the Norwalk virus on surfaces is an example of a public health environmental monitoring application.

Some embodiments may incorporate the use of a test tube 1, with a flexible device 10 divided into a plurality of segments, such as segments 16, 110, 120, 130, 140, 150, 160, 170, 180, and/or 190, that may be transverse to the longitudinal axis of the device, and which may contain reagents, such as reagents 210, 221, 222, 230, 240, 250, 260, 270, 280, and/or 290; as well as an analyzer, that may have a plurality of compression members, such as actuators 312, 322, 332, 342, 352, 362, 372, 382, and/or 392, clamps, such as clamps 310, 320, 330, 340, 350, 360, 370, 380, and/or 390, and blocks, for example 314, 344, and/or 394 (others unnumbered for simplicity); opposing the actuators and clamps, to process a sample. Various combinations of these actuators, clamps, and/or blocks may be used to effectively clamp the device closed thereby segregating fluid. In exemplary embodiments, at least one of the actuators or blocks may have a thermal control element to control the temperature of a device segment for sample processing. The sample processing apparatus can further have at least one magnetic field source 430 capable of applying a magnetic field to a segment. The sample processing apparatus can further have a detection device 492, such as photometer or a CCD, to monitor a reaction taking place or completed within the device.

Fluid can be driven through a flow-channel by compressing the device with a centrally positioned actuator, and its flanking clamps if any, to form a flow channel with a gap of about 1 to about 500 μm, preferably about 5 to about 500 μm through each segment. The adjacent actuators gently compress the adjacent segments in liquid communication with the flow-channel to generate an offset inner pressure to ensure a substantially uniform gap of the flow channel. The two flanking actuators can then alternatively compress and release pressure on the device on their respective segments to generate flow at a controlled flow rate. Optional flow, pressure, and/or force sensors may be incorporated to enable closed-loop control of the flow behavior. The flow-channel process can be used in washing, enhancing the substrate binding efficiency, and detection.

A particle immobilization and re-suspension process can be used to separate the particles from the sample liquid. The magnetic field generated by a magnetic source may be applied to a segment containing a magnetic particle suspension to capture and immobilize the particles to the tube wall.

An agitation process can be used during the capturing process. In another embodiment, a flow-channel can be formed in the segment with the applied magnetic field, and magnetic particles can be captured in the flow to increase the capturing efficiency. To resuspend immobilized particles, the magnetic field may be turned off or removed, and an agitation or flow-channel process can be used for re-suspension.

Embodiments of the present invention will be further described in the following examples, which do not limit the scope of the invention described in the claims.

### EXAMPLES

### Example 1: Large Stokes Shift Fluorescent Dyes

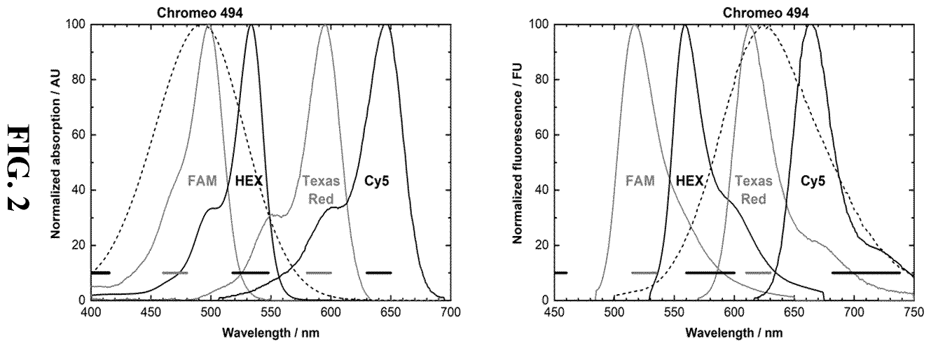

The concept of multiplexing PCR with LSS dyes was demonstrated by developing a panel for sexually transmitted infections (STI) for the Cobas® Liat® system. Two fluorogenic PCR probes (TaqMan probes) were prepared from commercially available LSS dyes; the Dy396XL “Mega-Stokes” dye (Dyomics, GmbH, Jena, Germany) and the Chromeo 494 dye (Active Motif, Carlsbad, CA, U.S.A.). FIG. 2 summarizes the excitation and emission spectra for four standard fluorescent dyes and Dy396XL (392 nm/572 nm), which can be excited in the UV channel and with readout occurring in the green channel. Likewise FIG. 3 summarizes a similar setup with Chromeo 494 dye (494 nm/628 nm), which can be excited with light from the blue channel and with readout occurring in the amber and/or red channel.

### Example 2: Multiplex PCR with LSS Dyes on the Cobas® Liat System

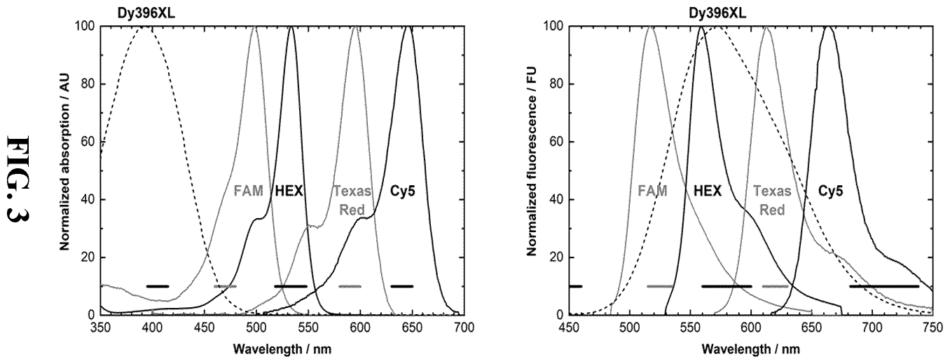

With the introduction of LSS dyes (Dy395XL and Chromeo494), the multiplexing level on the Cobas® Liat® system was increased from four detection channels (bb, gg, aa, rr) to six detection channels (adding ug and br). Table 2 provides an example for a 6-channel Cobas® Liat® CT/NG/TV/MG test. The LSS dyes, Dy395XL and Chromeo494, were used to label the MG and NG probes, respectively. FIG. 4 summarizes real-time PCR growth curves for six TaqMan probes in the Cobas® Liat® CT/NG/TV/MG test.

TABLE 2

| | Fluorescent | Detection |
|---|---|---|
| Target | dye | channel |
| Mycoplasma genitalium (MG) | Dy395XL | ug |
| Chlamydia trachomatis (CT) | FAM | bb |
| Trichomonas vaginalis (CT) | Chromeo494 | br |
| Internal process control (IC) | HEX | gg |
| Neisseria gonorrhoeae (NG) | CFR610 | aa |
| target 1 | | |
| Neisseria gonorrhoeae (NG) | Quasar670 | rr |
| target 2 | | |

In the 6-channel Cobas® Liat® CT/NG/TV/MG test feasibility of combining the four conventional channels (FAM, HEX, CFR610, and Quasar670) with the ug and br channels (Dy395XL and Chromeo494 respectively) was demonstrated (Table 3). After cross-talk correction, no signals were observed without corresponding targets.

TABLE 3

Fluorescence Amplitude

| Target | ug | bb | br | gg | aa | rr |
|---|---|---|---|---|---|---|
| MG | 1.07 | 0.01 | 0.00 | −0.01 | 0.00 | 0.00 |
| CT | 0.00 | 4.09 | 0.00 | 0.02 | −0.01 | 0.00 |
| TV | 0.01 | 0.00 | 1.66 | 0.02 | 0.01 | 0.00 |
| IC | −0.01 | 0.01 | −0.01 | 0.98 | 0.00 | 0.00 |
| NG | 0.00 | 0.01 | 0.01 | 0.02 | 12.72 | 3.41 |

### Example 3: Comparison of Dy395XL and Dy396XL

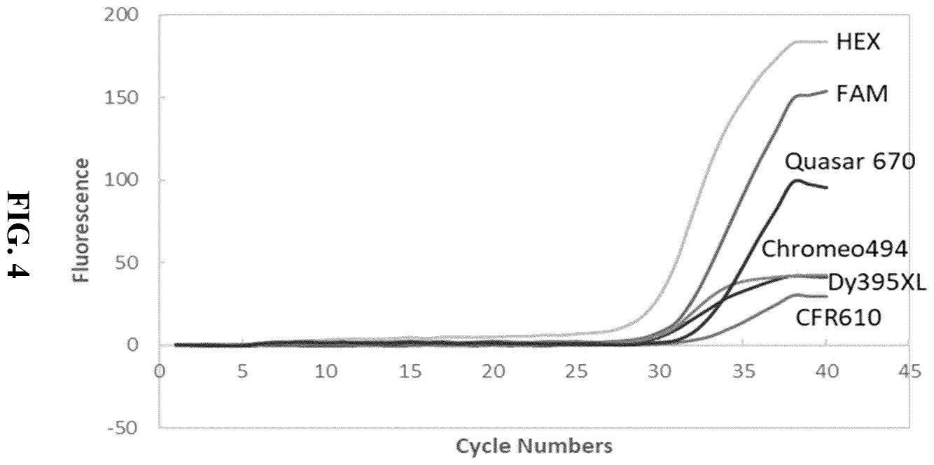

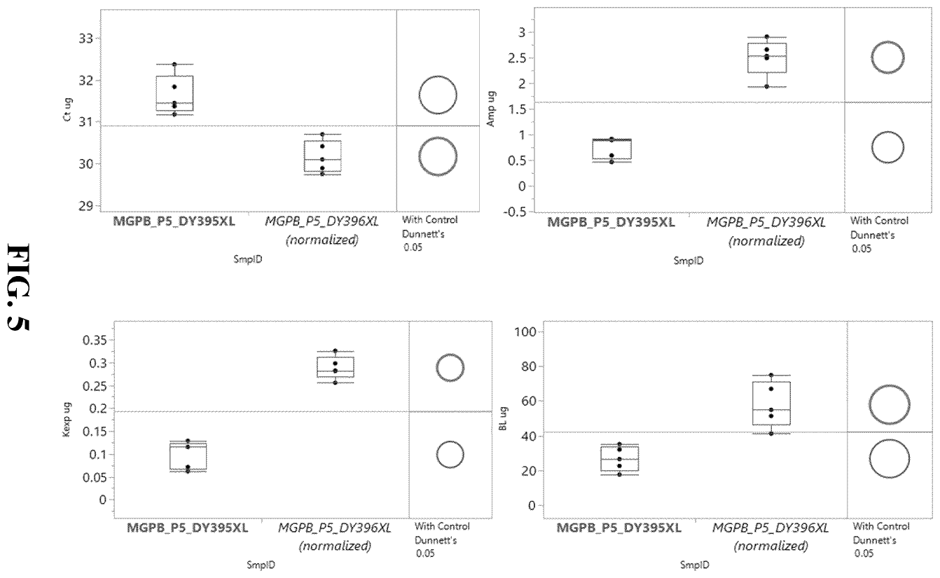

Further functional and manufacturability assessment of the LSS dyes showed that the fluorescence intensity of Dy395XL is fairly weak (c=20,600M⁻¹ cm⁻¹), and coupling with DNA cannot be scaled up due to exceptional low solubility in water miscible organic solvents and problematic purification. A Dy395XL analog with higher extinction coefficient (c=26,600M⁻¹ cm⁻¹), Dy396XL, showed feasible manufacturability of coupling with DNA. FIGS. 5-6 show performance comparisons of two MG probes each labeled with either Dy395XL or Dy396XL. The baseline of MG-Dy396XL probes was 1.5-2.5 fold higher than that of MG-Dy395XL probes. The statistical analysis showed that performance of the MG-Dy396XL probes was better than MG-Dy395XL probes in terms of Ct and amplitude (earlier Ct and higher amplitude). The higher Kexp values (slope of exponential phase) of MG-Dy396XL probes also suggested more robust growth curves for MG-Dy396XL probes.

### Example 4: Experimental Setup of Multiplex PCR with LSS Dyes and TAGS Technology on the LightCycler® 480 Analyzer

The following examples show that the concept of extended optical multiplexing with LSS dyes can be combined with other multiplexing methods. PCR with TAGS (Temperature assisted generation of signal) technology is based on a DNA probe architecture that allows discrimination of multiple targets in the same optical channel by measuring the fluorescence at different temperatures (as disclosed in U.S. Patent Publication No. 2018/0073064 and incorporated by reference herein in its entirety). Thermal multiplexing based on TAGS can be paired with optical multiplexing, provided that the fluorescence signal strength of the LSS dye remains stable at temperatures up to 100° C.

Multiplex PCR with the TAGS technology model system with three thermal channels (TC) has been built to demonstrate higher-order multiplexing. For TC1, standard TaqMan probes containing a 5′-fluorophore and an internal BHQ-2 quencher were used, whereas TC2 and TC3 employed tagged TAGS probes. The tagged probes were composed of a target specific DNA sequence, carrying a 5′-BHQ-2 fluorescence quencher, and a covalently bound “R-tag” sequence that is specific to the respective thermal and optical channel. The R-tag sequence carries a fluorescent dye and was made of unnatural L-DNA with a defined melting point to another complementary L-DNA strand that carries a second 3′-BHQ-2 fluorescence quencher (quenching oligonucleotide, “Q-tag”). The tagged probes for TC2 and TC3 only differ in the length of the L-DNA section. Regular TaqMan probes and tagged TAGS probes containing LSS dyes were prepared by introducing an amino-modification during solid phase DNA synthesis and post-synthetic labeling with the in situ activated carboxylic acid of the dye (as disclosed in U. S Patent Publication No. 2020/0017895 and incorporated by reference herein in its entirety). The oligonucleotides were purified with reverse phase chromatography, using triethylammonium acetate buffer and acetonitrile. The final tagged probes were purified by polyacrylamide gel electrophoresis.

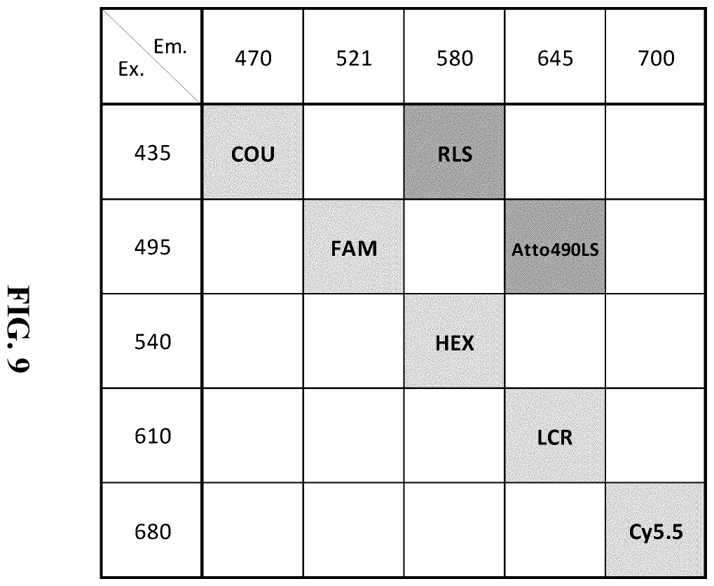

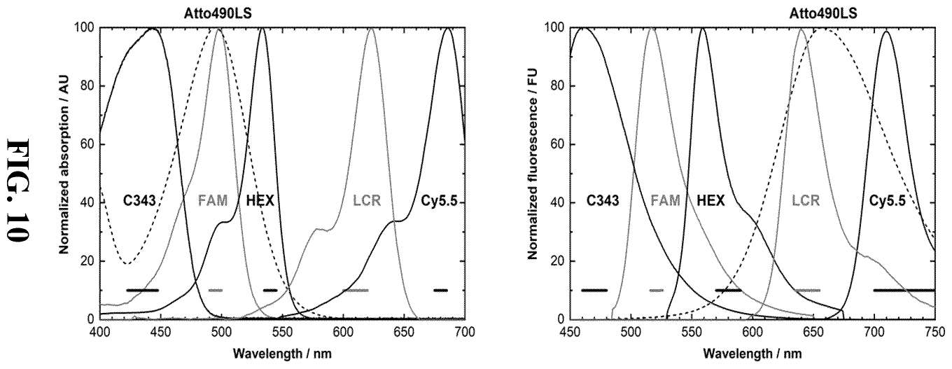

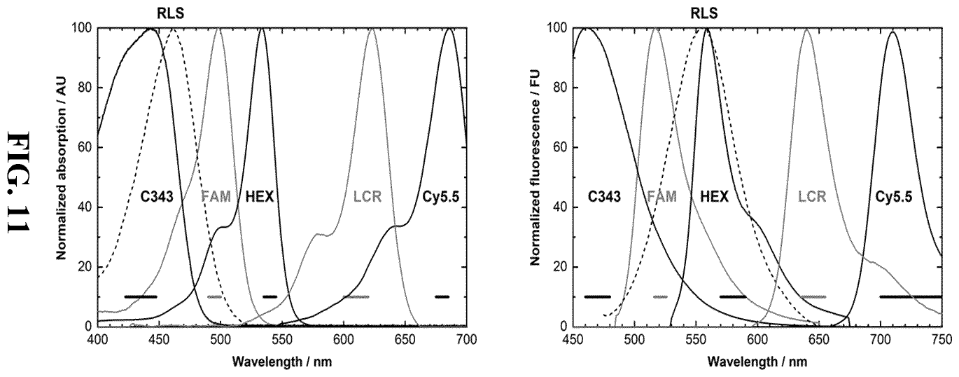

Two LSS dyes with thermostable fluorescence have been selected to demonstrate compatibility with TAGS technology; the commercially available ATTO 490LS dye (ATTO-TEC GmbH, Siegen, Germany) and another proprietary dye, that has been termed RLS. On the LightCycler® and Cobas® x800 systems, ATTO 490LS (496 nm/661 nm) can be excited in the FAM excitation channel (495 nm), with readout occurring in the LCR emission channel. (645 nm). The RLS dye (468 nm/553 nm) can be excited with light from the COU excitation channel (435 nm) and with readout occurring in the HEX emission channel (580 nm). The optical channel assignment matrix for the five standard dyes (COU, FAM, HEX, LCR, Cy5.5) and two LSS dye channels (ATTO 490LS and RLS) on the 5-color LightCycler® 480 and the Cobas® x800 analyzer is given in FIG. 9. An overview on the excitation and emission spectra for the five standard fluorescent dyes in combination with ATTO 490LS and RLS dye is shown in FIG. 10 and FIG. 11 respectively.

### Example 5: Multiplex PCR with Atto490LS Dye and TAGS Technology

In this example PCR reactions with thermal multiplexing and detection in an LSS optical channel were performed. The branched probes were incubated with quenching oligonucleotide at 1:20 molar ratio. The mixtures were typically cycled in 50 μL reactions that contained 60 mM Tricine, 120 mM potassium acetate, 5.4% DMSO, 0.027% sodium azide, 3% glycerol, 0.02% Tween 20, 43.9 μM EDTA, 0.2 U/μL UNG, 400 μL dATP, 400 μM CTP, 400 μM dGTP, 800 μM dUTP, 3.3 mM manganese acetate, 0.9 U Z05 enzyme, 800 nM Q-tag, 400 nM of each primer, and 40 nM of branched probe. Cycle conditions resembling a typical PCR amplification reaction are shown in the following Table 4.

TABLE 4

| | | | Temperature | | |
|---|---|---|---|---|---|
| Step | Description | Cycle No. | (° C.) | Time | |
| 1 | Sterilization | 1 | 50 | 2 | min. |
| | | | 94 | 5 | s |
| 2 | RT | | 55 | 2 | min. |
| | | | 60 | 6 | min. |
| | | | 65 | 4 | min. |
| | | | 95 | 5 | s |
| 3 | Dark cycles | 5 | 55 | 30 | s |
| | (no data | | 80 | 5 | s |
| | acquisition) | | 95 | 5 | s |
| 4 | TaqMan cycles | 45 | 58 | 25 | s |
| | | | 80 | 5 | s |
| | | | 91 | 5 | s |
| 5 | cooldown | | 40 | 2 | min. |

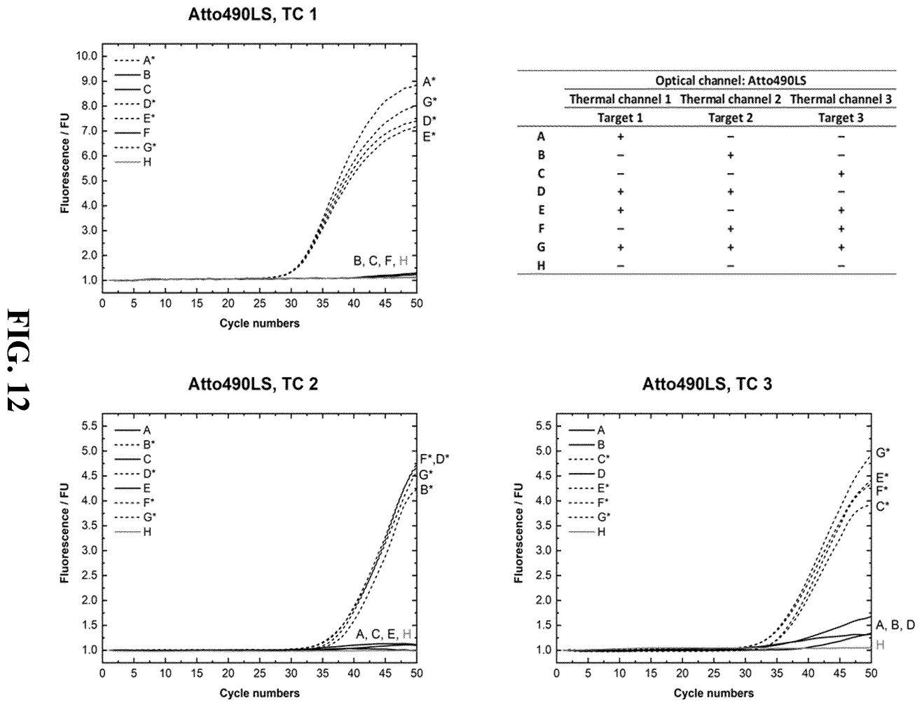

FIG. 12 shows the real-time PCR growth curves in the ATTO 490LS channel across three thermal channels. The data was generated on a LightCycler® 480 analyzer. TC1 is based on a standard TaqMan probe with ATTO 490LS dye and fluorescence readings at 58° C. TC2 and TC3 are based on tagged TAGS probe designs with the ATTO 490LS dye, that generate fluorescence readings at 80° C. and 91° C. respectively. All possible target combinations across the three thermal channels were tested in eight individual PCR reactions (Samples A-H). Growth curves for each thermal channel where a positive PCR signal was expected were marked with an asterisk. As expected, signals were only obtained where target was present. No signals were observed without corresponding targets. For the higher thermal channels, slight positive or negative slopes in the absence of target were caused by non-optimized thermal cross-talk correction for the fluorescent dye. The concentration of target was 1000 cp/reaction

### Example 6: Multiplex PCR with RLS Dyes and TAGS Technology

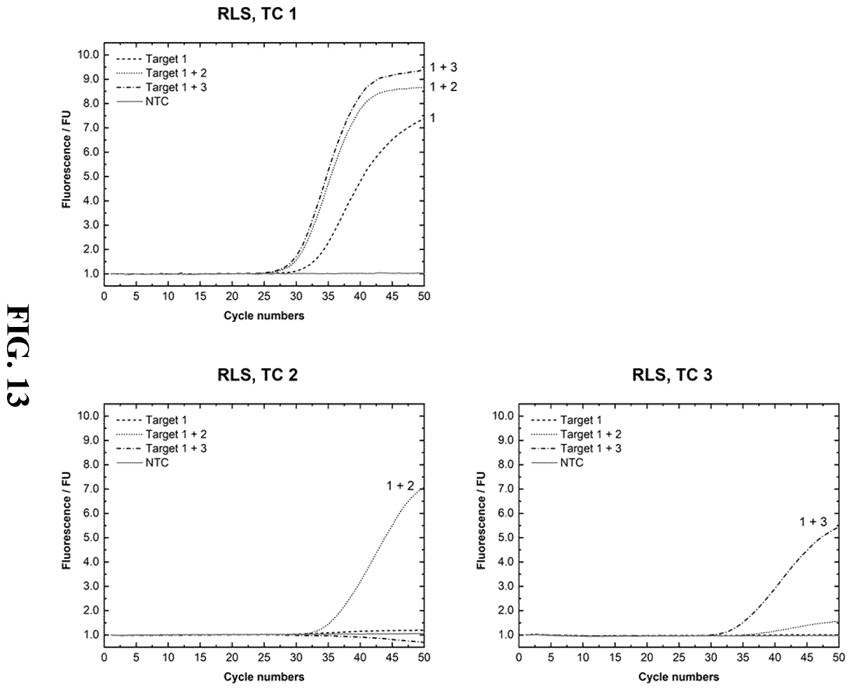

A similar experiment to the one described in Example 5 was performed, except that fluorescence detection was performed in a different LSS channel using a proprietary LSS dye termed RLS. As before, signals were only obtained where target was present. Notably, ATTO 490LS and RLS occupy two separate LSS channels. The crosstalk between the standard optical channels and the LSS dye channels was negligible, indicating that the ATTO 490LS and RLS can be used simultaneously. FIG. 13 shows the corresponding real-time PCR growth curves.

### Conclusions

The examples above demonstrate that with the introduction of LSS dyes, the multiplexing level on the LightCyler® and the Cobas® x800 systems can be increased from five conventional detection channels (COU, FAM, HEX, LCR, Cy5.5) to at least seven detection channels (adding ATTO 490LS and RLS). By combining multiplexing with LSS dyes and TAGS multiplexing with three temperature channels as demonstrated herein, the detection of 21 individual targets becomes possible.

In principle, higher-order optical multiplexing is compatible with any fluorescence-based PCR platform, if the fluorescence properties of the dyes match the optical filters of the instrument. In this way, no changes to instrument hardware are required. By the successful application of said concepts on two different PCR systems, Cobas® Liat and the LightCycler® 480 (which is directly transferrable to the Cobas® x800 systems), the platform independency of the optical multiplexing technology has also been shown.

1. A method for detecting at least two target nucleic acid sequences in a sample comprising the steps of:
(a) contacting said sample suspected of containing said at least two target nucleic acid sequences in a single reaction vessel with:
i. a first pair of oligonucleotide primers with nucleotide sequences that are complementary to each strand of a first target nucleic acid sequence, and a second pair of oligonucleotide primers with nucleotide sequences that are complementary to each strand of a second target nucleic acid sequence;
ii. a first oligonucleotide probe comprising a nucleotide sequence at least partially complementary to the first target nucleic acid sequence and annealing within the first target nucleic acid sequence bounded by the first pair of oligonucleotide primers, wherein said first oligonucleotide probe is labeled with a large stokes shift (LSS) fluorescent dye capable of generating a detectable signal and with a first quencher moiety capable of quenching the detectable signal generated by the LSS fluorescent dye, wherein the LSS fluorescent dye is separated from the first quencher moiety by a nuclease susceptible cleavage site;
iii a second oligonucleotide probe comprising a nucleotide sequence at least partially complementary to the second target nucleic acid sequence and annealing within the second target nucleic acid sequence bounded by the second pair of oligonucleotide primers, wherein said second oligonucleotide probe is labeled with a small stokes shift (SSS) fluorescent dye capable of generating a detectable signal and with a second quencher moiety capable of quenching the detectable signal generated by the SSS fluorescent dye, wherein the SSS fluorescent dye is separated from the second quencher moiety by a nuclease susceptible cleavage site, and wherein the SSS fluorescent dye has an absorption peak maximum significantly different from and an emission peak maximum similar to respective peak maxima of the LSS fluorescent dye on the first oligonucleotide probe, wherein the significant difference is at least 80 nanometer in wavelength;
(b) amplifying the first and second target nucleic acid sequences by polymerase chain reaction (PCR) using a nucleic acid polymerase having 5′ to 3′ nuclease activity such that during an extension step of each PCR cycle, the 5′ to 3′ nuclease activity of the nucleic acid polymerase allows cleavage and separation of the LSS fluorescent dye from the first quenching moiety on the first oligonucleotide probe, and cleavage and separation of the SSS fluorescent dye from the second quenching moiety on the second oligonucleotide probe;
(c) measuring the detectable signal from the LSS fluorescent dye by excitation at or near the wavelength of the absorption peak maximum of the LSS fluorescent dye and measuring the detectable signal from the SSS fluorescent dye by excitation at or near the wavelength of the absorption peak of the SSS fluorescent dye;
(d) repeating steps (b) and (c) in multiple PCR cycles to produce desired quantity of amplification products from the first and second target nucleic acid sequences;
(e) detecting the presence of the first target nucleic acid sequence from the signals detected from the LSS fluorescent dye and the presence of the second target nucleic acid sequence from the signals detected from the SSS fluorescent dye.

2. The method of claim 1, wherein the difference between the absorption peak maximum of the LSS fluorescent dye and the absorption peak maximum of the SSS fluorescent dye is greater than 80 nanometers in wavelength.

3. The method of claim 2, wherein the difference between the absorption peak maximum of the LSS fluorescent dye and the absorption peak maximum of the SSS fluorescent dye is greater than 100 nanometers in wavelength.

4. The method of any one of claims 1 to 3, wherein the LSS fluorescent dye is selected from the group consisting of: ALEXA FLUOR 430, ATTO 430LS, ATTO 490LS, ATTO 390LS, CASCADE YELLOW, CF350, CHROMEO 494, CYTO 500 LSS, CYTO 510 LSS, CYTO 514 LSS, CYTO 520 LSS, DAPOXYL, DY 480XL, DY 481XL, DY 485XL, DY 510XL, DY 511XL, DY 520XL, DY 521XL, DY 601XL, DY 350XL, DY 360XL, DY 370XL, DY 375XL, DY 380XL, DY 395XL, DY 396XL, DYLIGHT 515-LS, DYLIGHT 485-LS, DYLIGHT 510-LS, DYLIGHT 521-LS, FURA 2, INDO 1, KROME ORANGE, LUB 04, LUCIFER YELLOW, NBD X, NILE RED, PULSAR 650, PYMPO, STAR 440SXP, STAR 470SXP, STAR 520SXP, VIOGREEN, CF 350, SETAU 405, and PACIFIC ORANGE.

5. The method of claim 4, wherein the LSS fluorescent dye is selected from DY 396XL or CHROMEO 494.

6. The method of any one of claims 1 to 4, wherein the LSS fluorescent dye has a fluorescence signal strength that remains stable at temperatures up to 100° C.

7. The method of claim 6, wherein the LSS fluorescent dye is ATTO 490LS.

8. The method of any one of claims 1 to 7, wherein the first oligonucleotide probe, the second oligonucleotide probe or both the first oligonucleotide probe and the second oligonucleotide probe is a tagged probe compatible with the TAGS technology.

9. The method of any one of claims 1 to 8, wherein the reaction vessel is a tubule, comprising
(i) a proximal end having an opening through which a sample is introducible;
(ii) a distal end; and
(iii) at least a first segment containing at least one nucleic acid extraction reagent, a second segment distal to the first segment and containing a wash reagent, and a third segment distal to the second segment and containing one or more amplification reagents, each of said segments being:
(A) defined by the tubule;
(B) fluidly isolated, at least in part, by a fluid-tight seal formed by a bonding of opposed wall portions of the tubule to one another such that:
(1) the seal is broken by application of fluid pressure on a segment that is fluidly isolated in part by the seal; and
(2) the seal is capable of being clamped where the opposed wall portions of the tubule are bonded, without breaking the seal, to prevent the seal from being broken by application of fluid pressure on a segment that is fluidly isolated in part by the seal;
(C) so expandable as to receive a volume of fluid expelled from another segment; and so compressible as to contain substantially no fluid when so compressed;
(iv) a cap for closing the opening, the cap containing a chamber in fluid communication with the tubule, and the cap permitting free escape of gasses but retaining all liquid volumes and infectious agents in the tube;
(v) a rigid frame to which the tubule's proximal and distal ends are held; and
(vi) an integral tubule tensioning mechanism or an attachment of the tubule to the frame that pulls the tubule sufficiently taut so as to facilitate compression and flattening of the tubule.

10. A method for detecting at least two target nucleic acid sequences in a sample comprising the steps of:
(a) contacting said sample suspected of containing said at least two target nucleic acid sequences in a single reaction vessel with:
i. a first pair of oligonucleotide primers with nucleotide sequences that are complementary to each strand of a first target nucleic acid sequence, and a second pair of oligonucleotide primers with nucleotide sequences that are complementary to each strand of a second target nucleic acid sequence;
ii. a first oligonucleotide probe comprising a nucleotide sequence at least partially complementary to the first target nucleic acid sequence and annealing within the first target nucleic acid sequence bounded by the first pair of oligonucleotide primers, wherein said first oligonucleotide probe is labeled with a large stokes shift (LSS) fluorescent dye capable of generating a detectable signal and with a first quencher moiety capable of quenching the detectable signal generated by the LSS fluorescent dye, wherein the LSS fluorescent dye is separated from the first quencher moiety by a nuclease susceptible cleavage site;
iii a second oligonucleotide probe comprising a nucleotide sequence at least partially complementary to the second target nucleic acid sequence and annealing within the second target nucleic acid sequence bounded by the second pair of oligonucleotide primers, wherein said second oligonucleotide probe is labeled with a small stokes shift (SSS) fluorescent dye capable of generating a detectable signal and with a second quencher moiety capable of quenching the detectable signal generated by the SSS fluorescent dye, wherein the SSS fluorescent dye is separated from the second quencher moiety by a nuclease susceptible cleavage site, and wherein the SSS fluorescent dye has an emission peak maximum significantly different from and an absorption peak maximum similar to respective peak maxima of the LSS fluorescent dye on the first oligonucleotide probe, wherein the significant difference is at least 80 nanometer in wavelength;
(b) amplifying the first and second target nucleic acid sequences by polymerase chain reaction (PCR) using a nucleic acid polymerase having 5′ to 3′ nuclease activity such that during an extension step of each PCR cycle, the 5′ to 3′ nuclease activity of the nucleic acid polymerase allows cleavage and separation of the LSS fluorescent dye from the first quenching moiety on the first oligonucleotide probe, and cleavage and separation of the SSS fluorescent dye from the second quenching moiety on the second oligonucleotide probe;
(c) measuring the detectable signal from the LSS fluorescent dye by excitation at or near the wavelength of the absorption peak maximum of the LSS fluorescent dye and measuring the detectable signal from the SSS fluorescent dye by excitation at or near the wavelength of the absorption peak of the SSS fluorescent dye;
(d) repeating steps (b) and (c) in multiple PCR cycles to produce desired quantity of amplification products from the first and second target nucleic acid sequences;
(e) detecting the presence of the first target nucleic acid sequence from the signals detected from the LSS fluorescent dye and the presence of the second target nucleic acid sequence from the signals detected from the SSS fluorescent dye.

11. The method of claim 10, wherein the difference between the emission peak maximum of the LSS fluorescent dye and the emission peak maximum of the SSS fluorescent dye is greater than 80 nanometers in wavelength.

12. The method of claim 11, wherein the difference between the emission peak maximum of the LSS fluorescent dye and the emission peak maximum of the SSS fluorescent dye is greater than 100 nanometers in wavelength.

13. The method of any one of claims 10 to 12, wherein the LSS fluorescent dye is selected from the group consisting of: ALEXA FLUOR 430, ATTO 430LS, ATTO 490LS, ATTO 390LS, CASCADE YELLOW, CF350, CHROMEO 494, CYTO 500 LSS, CYTO 510 LSS, CYTO 514 LSS, CYTO 520 LSS, DAPOXYL, DY 480XL, DY 481XL, DY 485XL, DY 510XL, DY 511XL, DY 520XL, DY 521XL, DY 601XL, DY 350XL, DY 360XL, DY 370XL, DY 375XL, DY 380XL, DY 395XL, DY 396XL, DYLIGHT 515-LS, DYLIGHT 485-LS, DYLIGHT 510-LS, DYLIGHT 521-LS, FURA 2, INDO 1, KROME ORANGE, LUB 04, LUCIFER YELLOW, NBD X, NILE RED, PULSAR 650, PYMPO, STAR 440SXP, STAR 470SXP, STAR 520SXP, VIOGREEN, CF 350, SETAU 405, and PACIFIC ORANGE.

14. The method of claim 13, wherein the LSS fluorescent dye is DY 396XL or CHROMEO 494.

15. The method of any one of claims 10 to 13, wherein the LSS fluorescent dye has a fluorescence signal strength that remains stable at temperatures up to 100° C.

16. The method of claim 15, wherein the LSS fluorescent dye is ATTO 490LS.

17. The method of any one of claims 10 to 16, wherein the first oligonucleotide probe, the second oligonucleotide probe or both the first oligonucleotide probe and the second oligonucleotide probe is a tagged probe compatible with the TAGS technology.

18. The method of any one of claims 10 to 17, wherein the reaction vessel is a tubule, comprising
(i) a proximal end having an opening through which a sample is introducible;
(ii) a distal end; and
(iii) at least a first segment containing at least one nucleic acid extraction reagent, a second segment distal to the first segment and containing a wash reagent, and a third segment distal to the second segment and containing one or more amplification reagents, each of said segments being:
(A) defined by the tubule;
(B) fluidly isolated, at least in part, by a fluid-tight seal formed by a bonding of opposed wall portions of the tubule to one another such that:
(1) the seal is broken by application of fluid pressure on a segment that is fluidly isolated in part by the seal; and
(2) the seal is capable of being clamped where the opposed wall portions of the tubule are bonded, without breaking the seal, to prevent the seal from being broken by application of fluid pressure on a segment that is fluidly isolated in part by the seal;
(C) so expandable as to receive a volume of fluid expelled from another segment; and so compressible as to contain substantially no fluid when so compressed;
(iv) a cap for closing the opening, the cap containing a chamber in fluid communication with the tubule, and the cap permitting free escape of gasses but retaining all liquid volumes and infectious agents in the tube;
(v) a rigid frame to which the tubule's proximal and distal ends are held; and
(vi) an integral tubule tensioning mechanism or an attachment of the tubule to the frame that pulls the tubule sufficiently taut so as to facilitate compression and flattening of the tubule.
